(19) United States
(12) Patent Application Publication
(10) Pub. No.: US 2006/0211071 A1
(43) Pub. Date: Sep. 21, 2006

(54) Device for aggregating, imaging and analyzing thrombi and a method of use

(75) Inventors: Patrick Andre, San Mateo, CA (US); Hans Luedemann, Bolton, MA (US); David Phillips, San Mateo, CA (US); Golnaz Shapurian, Newton, MA (US); Craig Muir, Westford, MA (US)

Correspondence Address: ATTN: Patent Group;COOLEY GODWARD LLP, The Bowen Building, 875 15th Street, NW, Suite 800, Washington, DC, 20005-2221, US

(73) Assignee: Millennium Pharmaceuticals, Inc.

(21) Appl. No.: 11/302,210

(22) Filed: Dec. 14, 2005

Related U.S. Application Data
(60) Provisional application No. 60/635,659, filed on Dec. 14, 2004.

(51) Int. Cl. C12Q 1/56; G06F 19/00; G06K 9/00; C12M 1/34

(52) U.S. Cl. 435/13; 435/287.1; 702/19; 382/128

(57) ABSTRACT

An instrument for capturing an image of thrombus formation, blood coagulation, recruitment of circulating inflammatory or tumour cells in a blood sample. The instrument comprises a member defining a channel therethrough, a fluid handling assembly that permits the blood sample to move through the channel at a flow rate, and an imaging assembly including a microscopy device. The imaging assembly is disposed relative to the channel so as to capture light rays defining the image of thrombus formation in the channel.

This application claims the benefit of U.S. Provisional Application Ser. No. 60/635,659 filed Dec. 14, 2004, which is incorporated by reference herein in its entirety.

### FIELD OF INVENTION

The invention relates generally to a device and method for producing and analyzing blood deposits to obtain a blood deposit profile. More particularly, to a device and system for analyzing the kinetics of thrombosis (platelet adhesion, thrombus growth, stability and reversal), blood coagulation and biological behavior of blood sample constituents (leukocytes and circulating tumor cells. The assays and analytical tools embodied in the systems enable novel and clinically relevant information for use in characterizing modifiers of constituent responses as affected by genetic, experimental and/or pharmacological modulation and or variation.

### DESCRIPTION OF RELATED ART

Evaluation of the thrombotic process in humans has been achieved using different approaches. One way is the use of a platelet aggregometer. Using different platelet agonists, platelet aggregometers study the aggregation process involving ADP, collagen, vWF, and thrombin pathways, for example. This device requires the use of anti-coagulated blood; however, all anti-coagulants affect thrombotic process and therefore can cause misreading of the anti-thrombotic properties of anti-platelet drugs. Also, platelet rich plasma or washed platelets need to be prepared using sequential centrifugation, which can require processing up to one hour or more before the thrombotic profile is known. The platelet rich plasma is further known to activate platelets and makes the method less informative of underlying biology and pharmacological response. This device is based on platelet-platelet interactions occurring under low shear conditions (venous shear rate) and no real indications of the adhesion process are obtained.

Another way is to evaluate the thrombotic process is to use the Dade Behering/IDEO-Baxter Diagnostics, PFA-100 Platelet Function analyzer in which the process of platelet adhesion and aggregation following a vascular injury is simulated in vitro. Membranes consisting of Collagen/Epinephrine (CEPI) and Collagen/Adenosine-5′-diphosphate (CADP) and the high shear rates generated under standardized flow conditions, result in platelet attachment, activation and aggregation, building a stable platelet plug at the aperture. The time required to obtain full occlusion of the aperture is reported as the closure time (CT) in seconds. The test is sensitive to platelet adherence and aggregation abnormalities and allows the discrimination of aspirin-like defects and intrinsic platelet disorder. The CEPI membrane is used to detect platelet dysfunction induced by intrinsic platelet defects (vWD, drug effects, etc.) Abnormalities result in prolongation of CT>175 seconds. Follow-up testing using the CADP membrane enables the discrimination of aspirin effects. An assay of samples of anti-coagulated whole blood produces results in less than thirty minutes following blood puncture, however, there can be drawbacks to this analyzer. Like the platelet aggregometer, this analyzer also requires the use of anti-coagulated blood. It measures time for occlusion under high shear rates, but differentiation cannot be made between an anti-adhesive and anti-aggregatory treatment. Nor does this system allow for a precise study of the level of inhibition achieved by anti-thrombotic drugs, the kinetics of thrombosis and the antithrombotic profiles of therapeutic agents and their combination.

Another way to monitor the thrombotic process is to use an Ultegra Rapid Platelet Function Assay (RPFA), which is an automated turbidimetric, whole blood assay to assess platelet function based on the ability of activated platelets to bind to fibrinogen coated beads. The detection well of the Ultegra RPFA-TRAP Cartridge contains all of the necessary reagent to perform this analysis. Within the well is an activator that induces the platelet to change the conformation of the GPIIb/IIIa receptor to a form that binds fibrinogen. Additionally, the detection well also contains fibrinogen-coated microbeads that bind to activated GPIIb/IIIa receptors. The GPIIb/IIIa receptors on activated platelets will bind to the fibrinogen-coated microbeads and cross link to other microbeads resulting in a clearing of the bead and platelets within the detection well. The analyzer uses light transmittance to measure the rate at which this clearing occurs. If the GPIIb/IIIa receptors on the platelet are inhibited, for instance, by abciximab, there will be minimal binding of the microbeads with activated platelets, since the GPIIb/IIa receptor sites are blocked by the drug and cannot bind to the fibrinogen coated beads. In this instance there will be minimal clearing of the sample and little change in the amount of light that is transmitted through the sample. This assay requires the use of anti-coagulated blood, it occults the shear-dependent effect and it does not give indication of the adhesion process, the kinetics of thrombosis and the mechanistic features of antothrombotic drugs.

Another device is of the type proposed in U.S. Pat. No. 5,662,107 to Sakariassen. This patent discloses a device and method for measuring thrombus formation tendency under simulated in vivo conditions. The blood is pumped at a constant flow through at least one flow channel that can be coated or made of a thrombogenesis-promoting material. The pressure differences between the pressures upstream and downstream of the thrombogenesis unit, due to a thrombus formed in the flow channel, is measured. The use of the flow device as a portable thrombosis screening device is prevented by two major limitations. The flow device in this patent is complex, requires assembly, and requires the use of a screw to seal the plates. To study different conditions of shear or thrombogenic surfaces, this patent proposes the use of different perfusion chambers in parallel. This patent discloses the use of computer assisted morphometry analysis of the thrombotic deposits based on the embedding of the thrombotic deposits in Epon, sectioning of the embedded rods, then quantification of the percentage of adhesion and thrombus size on semi-thin cross sections. Results are obtained after a minimum of two days. To expedite detection of the thrombotic process, the patent discloses a proposed measurement of the variations of the blood pressure as an indication of the thrombotic process. This device and method, however, is imprecise because of the inability to perform a dose response curve with anti-thrombotic agents, for example. Two sensors will need to be mounted upstream and downstream of the perfusion chamber, increasing the time to prepare the chamber. Also, there needs to be a recording device, a processor and a display in close proximity to the patient.

Also known in the art is the use of capillary tubes as the perfusion chamber. The cross-sectional dimension of the capillary tube are a limitation on the assay because the tubes, as presently configured, require a minimum volume of blood sample in order to run an assay. Specifically, capillary tubes have an inner diameter of about 400 microns.

What is needed is a device that will assay a blood sample and provide image data of thrombus formation and correlate the image data to thrombus volume and other quantifiable characteristics of the thrombus formation for use in modifying and measuring the efficacy of anti-thrombotic therapies in real time. Preferably, the device would permit kinetic study of a thrombus formation by capturing time-lapse images of the thrombus formation. Preferably, the device would produce and analyze the image data to give a rapid, for example less than thirty minutes, thrombotic profile, including both adhesion and aggregation parameters for one individual. The profile would preferably be sensitive to any of the possible anti-platelet and anticoagulant agents and their combination, and to inhibitors of leukocyte and tumor cells recruitment so that a patient's therapy can be monitored. Additionally, the device would provide for a self contained member or perfusion chamber in which to conduct the assay and hold the blood sample for safety and disposability. The perfusion chamber would preferably be minimized so as to reduce the volume of the requisite sample necessary for performing the assay. The device would preferably provide for a computer interface to control the fluid handling and imaging components of the instrument. The computer interface would also provide for a reporting display to communicate the results of the analysis. Finally, it would also be desirable to have the ability to use various thrombogenic surfaces at the same time to cover all the major anti-platelet therapies. The ability to run multiple simultaneous or parallel blood assays can provide for a way to rapidly generate and investigate a dose response curve for a given patient and antithrombotic agent therapy.

### SUMMARY OF THE INVENTION

Incorporated in its entirety by reference hereto is U.S. provisional patent application entitled, “Devices And Methods For Identifying And Treating Aspirin Non-Responsive Patients” assigned to Portola Pharamceuticals, Inc., filed on Dec. 14, 2004 having Ser. No. 60/636,744 and Townsend and Townsend and Crew, LLP Attorney Docket No. 022104-001310US.

The present invention provides an instrument for capturing the kinetics of thrombus formation, coagulation, leukocyte an tumor cell recruitment in a blood sample. In a preferred embodiment the instrument provides for generating a video of thrombus formation. The instrument comprises a member defining a channel therethrough, a fluid handling assembly that permits the blood sample to move through the channel at a flow rate, and an imaging assembly including a microscopy device. The imaging assembly is disposed relative to the channel so as to capture light rays defining the image of thrombus formation in the channel.

In another embodiment of the present invention, a system for quantifying thrombus formation from a digital data image of a blood sample comprises a digital read/write medium to load the digital data, a processor for converting the digital data to pixel data, and software having at least one algorithm for quantifying the thrombus formation using the pixel data.

In yet another embodiment of the present invention, a method of quantifying thrombus formation from a blood sample comprises providing a member having at least one channel, the channel includes at least one surface coated with a thrombogenic material. The method includes moving the blood sample through the channel so as to initiate thrombus formation upon the blood sample contacting the thrombogenic material, and imaging the thrombus formation by microscopy.

In another embodiment of the present invention a member for capturing thrombus formation comprises a body defining at least one channel therethrough, the channel has an inlet end and an outlet end. A transparent section of the body defines at least a portion of the channel, and the transparent portion comprises substantially a non-thrombogenic material. At least a portion of the transparent portion is coated with either a thrombogenic, a pro-coagulant, pro-inflammatory material or a chemoattractant/adhesive surface for circulating tumor cells.

In another embodiment of the present invention, provided is an instrument for capturing an image of thrombus formation in a member having a channel for moving a blood sample therethrough. The instrument comprises a socket configured to receive the member, a fluid handling assembly that permits the blood sample to move through the channel at a flow rate, and an imaging assembly including a microscopy device. The imaging assembling is disposed relative to the socket to permit the imaging assembly to capture an image of thrombus formation in the channel.

### BRIEF DESCRIPTION OF THE DRAWINGS

The accompanying drawings, which are incorporated herein and constitute part of this specification, illustrate an embodiment of the invention, and, together with the general description given above and the detailed description given below, serve to explain the features of the invention.

FIG. 1A is a schematic view of an instrument used in the aggregation of platelets to image and analyze thrombus formations;

FIG. 1B is a flowchart of an embodiment of operation of the instrument of FIG. 1A;

FIG. 1C is an illustrative embodiment of the instrument of FIG. 1A;

FIG. 1D is a schematic of another instrument used in the aggregation of platelets to produce thrombus formations and also used in the imaging and analysis of the formations;

FIG. 1E is a flowchart of an embodiment of operation of the instrument of FIG. 1D;

FIG. 1F is an illustrative embodiment of the instrument of FIG. 1D;

FIG. 1G is a preferred embodiment of a socket used in the instruments of FIGS. 1A and 1D;

FIG. 1H is a series of still images of thrombus formations produced by the instrument of FIG. 1A;

FIGS. 2A-C are cross-sectional views of various embodiments of a member used in the instrument of FIG. 1 to aggregate platelets and produce thrombus formations;

FIGS. 3A-3D are views of another preferred embodiment of the member;

FIGS. 3E-3G are a top and plan views of another preferred embodiment of the member;

FIG. 3H are top and plan views of another embodiment of the member in FIGS. 3E-G;

FIGS. 3I-3K are plan and perspective views of another preferred embodiment of the member;

FIGS. 3L-3M are perspective views of another preferred embodiment of the member in FIGS. 3I-3K;

FIG. 4 is a screen snapshot of an embodiment of a graphical user interface for use with the instrument of FIG. 1;

FIGS. 4A-4B are graphical representations correlating volume of thrombus formation to the image data produced by a preferred embodiment of the instrument;

FIG. 4C is a sample of the image data produced by a preferred embodiment of the instrument;

FIG. 4D is a graphic representation of change in mean pixel value over time produced by the instrument of FIG. 1A;

FIG. 5 is a schematic view of a control system for use with the instruments of FIGS. 1A and 1D;

FIG. 6A is a digital image of a sample using the method according to the present invention;

FIG. 6B is a background subtracted image of the digital image in FIG. 6A;

FIG. 6C is low-pass filtered image of the digital image in FIG. 6A;

FIG. 6D is a thrombus area calculated image of the sample from FIG. 6A;

FIG. 6E is a volume calculated image of the sample from FIG. 6A;

FIG. 6F is a perimeter calculated image of the sample from FIG. 6A;

FIGS. 7A-7C are illustrative frame by frame histogram plots of pixel intensity values generated by an algorithm according to the present invention;

FIGS. 7D-7G are temporal plots of pixel value histograms;

FIG. 8A is an illustrative pixel intensity plot according to the present invention;

FIGS. 8B-8H are illustrative quantifying plots of thrombus formation generated by the algorithm according to the present invention;

FIG. 9 is an illustrative histogram, first derivative, and second derivative functions of a binarized grayscale image generated by a second algorithm according to the present invention;

FIGS. 9A-9F are illustrative digital images generated by the second algorithm;

FIG. 9G is an illustrative frame by frame plot of thrombus volume growth/decay generated by the second algorithm;

FIGS. 10A-10C are the results of several anticoagulants and their effects on the antithrombotic activity of a P2Y₁₂ antagonist;

FIG. 11 is an illustration of the thrombosis profiler and an example of a thrombotic profile;

FIG. 12 is an illustration of how thrombus size is determined;

FIG. 13 are thrombotic profiles illustrating the effect of increasing shear on platelets;

FIG. 14 illustrates the reproducibility of thrombotic profiles between perfusion chambers for the same blood donor;

FIG. 15 are thrombotic profiles which illustrates that syk antagonist inhibits platelet adhesion, thrombus growth and thrombus stability on collagen;

FIG. 16 are thrombotic profiles which illustrates the effect of increasing concentration of Eptifibatide (a GP IIb/IIIa inhibitor) on the thrombotic process;

FIGS. 17A-17B are thrombotic profiles of an individual before and after Plavix therapy;

FIGS. 18A-18D summarizes the results of several P2Y₁₂ inhibition studies;

FIG. 19 are thrombotic profiles which illustrates that inhibiting syk tyrosine kinase contributes to thrombosis reversal;

FIG. 20 are the results of a sequential study evaluating the maximum peak (Fluorescence intensity/total area (μM²) reflecting thrombus height) of twenty healthy volunteers dosed with clopidogrel, aspirin and their combination;

FIG. 21 are the thrombotic profiles of a type II diabetic patient showing a lack of protection by plavix (plavix resistance) despite two 300 mg loading dose of plavix and daily use of aspirin, in whom a direct P2Y₁₂ antagonist confers antithrombotic activity;

FIG. 22 are mean thrombotic profiles of blood treated with enoxaparin and fXa inhibitor and perfused over a collagen+tissue factor coated matrix.

### DETAILED DESCRIPTION OF THE PREFERRED EMBODIMENTS

### I. THE INSTRUMENT

Instrument I

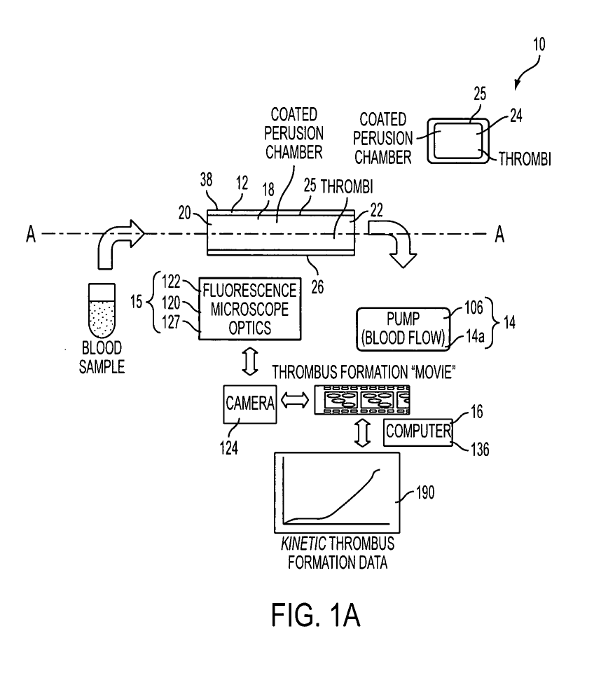

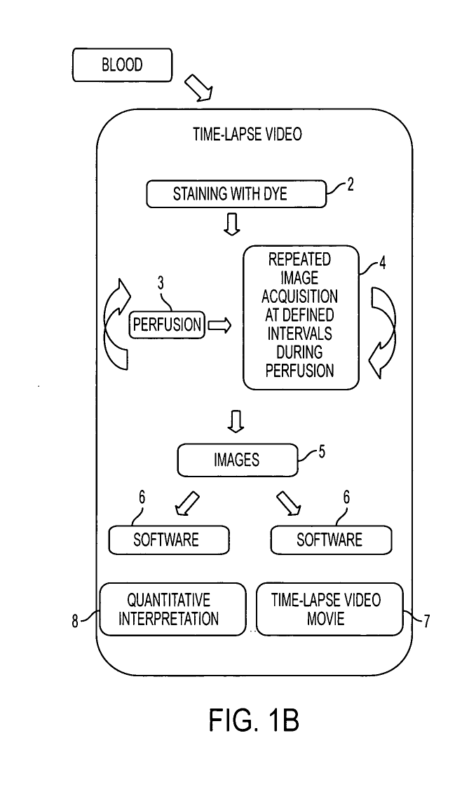

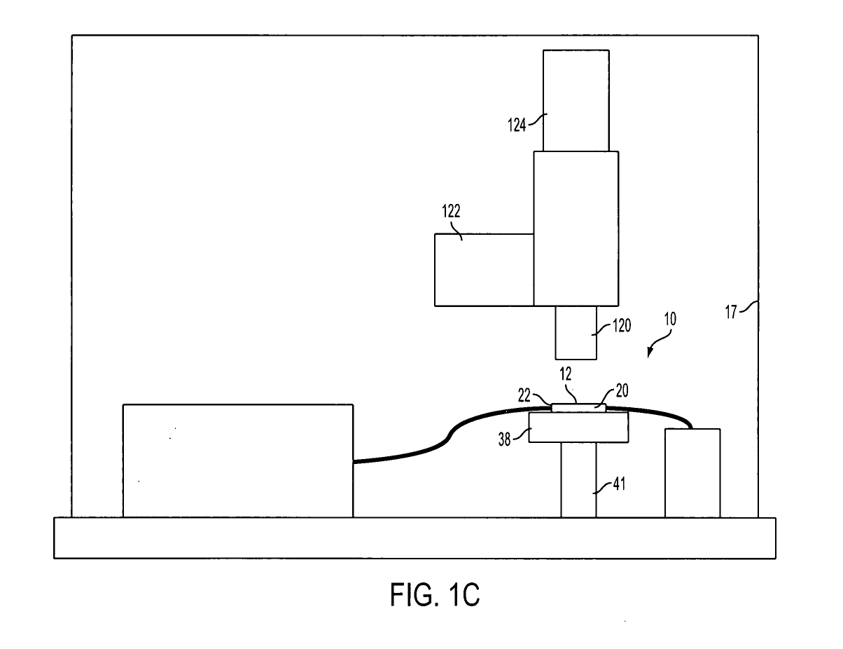

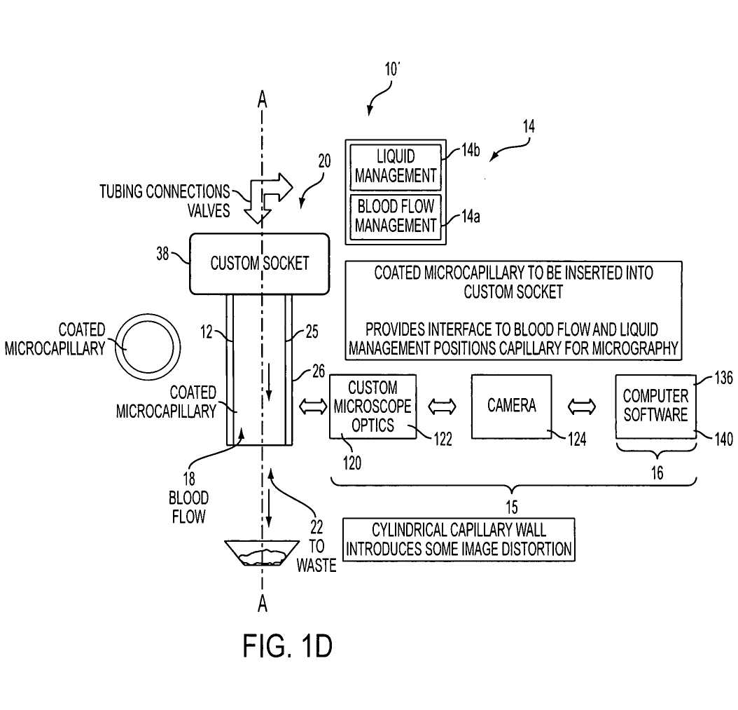

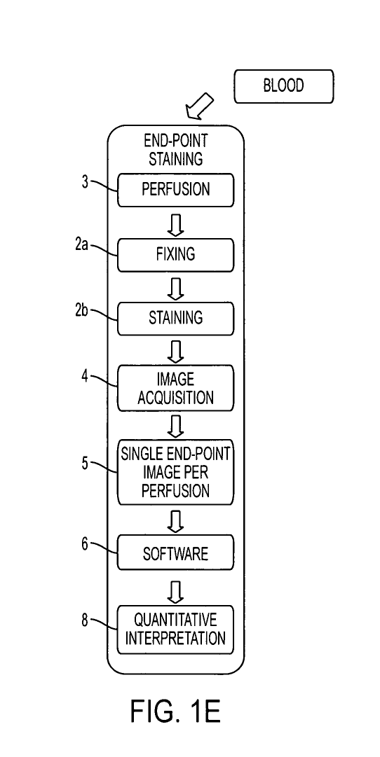

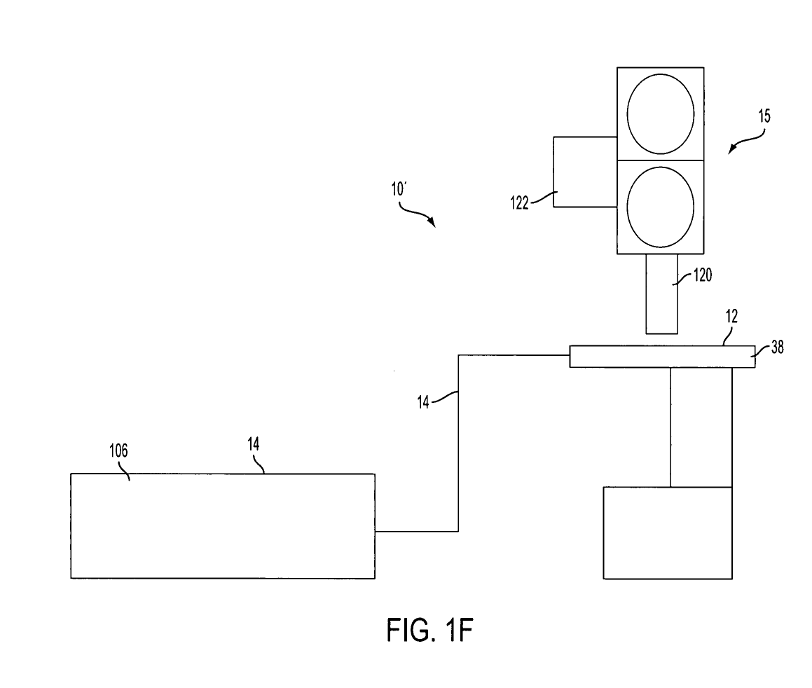

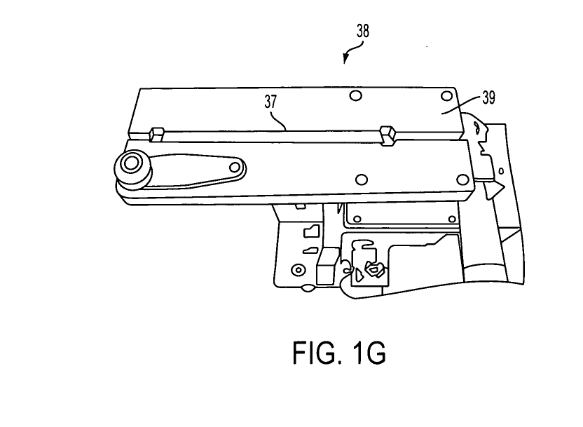

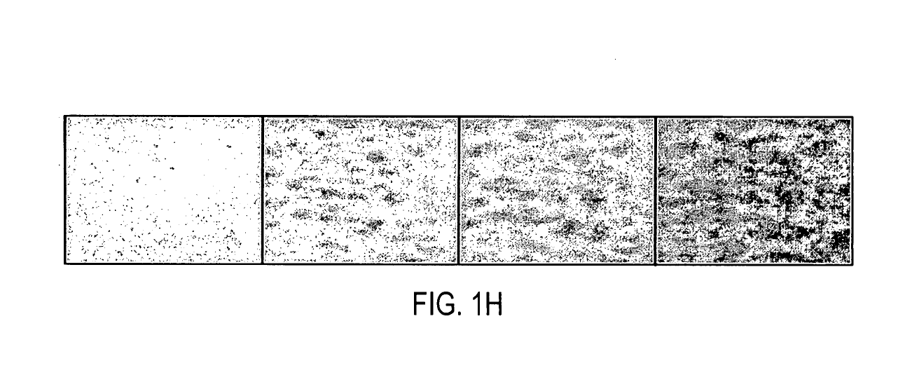

Shown in FIG. 1A is a schematic diagram of a preferred embodiment of an instrument 10, in the form of a kinetic aggregometer instrument for capturing a kinetic, moving or time-lapse image of thrombus formation, coagulation, leukocyte or tumor cell recruitment in a blood sample containing, for example, an anti-thrombotic agent. To image the thrombus formation, the instrument 10 uses microscopy and/or micro-videography techniques, and preferably light microscopy techniques. Shown in FIG. 1B is a flowchart of a preferred embodiment of operation of the instrument 10. Referring to both FIGS. 1A & 1B, the instrument 10 includes a member 12, a fluid handling assembly 14, an imaging assembly 15, and a data analyzer 16. According to box steps 2 and 3 in FIG. 1B, a sample of blood can be pre-treated with an imaging agent or fluorescent label and moved or perfused through member 12 by the fluid handling assembly 14 for a period of time so as to initiate thrombus formation within the member 12. Alternatively, the imaging agent can be added to the sample during the perfusion process. The imaging assembly 15 in box step 5 repeatedly images the developing thrombus formation within the member 12 during the perfusion using a camera 124 capable of motion capture. The imaging assembly 15 preferably uses light microscopy and/or micro-videography techniques with fluorescence illumination. The image can be preferably captured as time-lapsed digital image data and integrated over time to provide a movie or motion picture display of the evolving thrombus formation as is indicated by step boxes 6 and 7. In addition, the digital image data can be processed and correlated by analyzer 16 to quantify a temporal evolution of volume of thrombus formation or other quantifiable characteristics of thrombi formation, as is indicated by step boxes 6 and 8. This information can be useful in determining the real time efficacy of a given anti-thrombotic therapy using, for example: aspirin, P2Y₁₂ receptor targeted compounds and GPIIb/IIIa antagonists, Integrilin as well as other platelet-thrombus modulators, and can serve as feedback information to modifying the dosage of the therapy. The imaging assembly 15 can additionally include a non-imaging photodetector 127 that generates a signal in response to the fluorescence intensity of the thrombus formation. The signal can be used by the data analyzer 16 to correlate and quantify, in an alternate manner, the temporal evolution of the thrombus volume, in addition to other quantifiable characteristics of thrombus formation.

The Instrument II

Referring now to FIG. 1D is a schematic of an alternative embodiment of the instrument 10′ which can be configured for fixed imaging or “end-point measurement” of thrombi. Specifically, instrument 10′ is configured for imaging the thrombus formation at a fixed point in time, preferably at the conclusion of the thrombus formation process using light microscopy techniques. Shown in FIG. 1E is a flow chart of a preferred embodiment of operation the instrument 10′ in FIG. 1D.

Instrument 10′, like instrument 10 of FIG. 1A, can also generally include a member 12, a fluid handling assembly 14, an imaging assembly 15, and an analyzer 16. Referring to both FIGS. 1D and 1E, the fluid handling assembly 14 of instrument 10′ perfuses or moves a sample of blood through member 12 for a period of time so as to initiate thrombus formation within the member 12. The sample of blood can be subsequently treated with image enhancing agents that fix and stain the thrombus formation within the member 12, as is shown by step boxes 2a and 2b. The image enhancing agents can be delivered by the fluid handling assembly 14. The imaging assembly 15 can image the thrombus formation formed within the member 12 using microscopy techniques known to one of ordinary skill in the art, as indicated in step boxes 4 and 5. The imaging assembly 15 of instrument 10′ preferably uses light microscopy with K \overline{h}ler illumination. The imaging assembly 15 can additionally capture the image as digital image data using a camera 124. The digital image data can be further processed by analyzer 16 in order to determine the volume of thrombus formation and other quantifiable characteristics of thrombus formation, such as for example, height, area and perimeter of the thrombus formation.

### II. THE MEMBER

The member 12 is preferably configured for capturing the thrombus formation to be imaged and may be used in systems using either kinetic imaging or fixed end-point imaging of the thrombus formation.

Capillary Tube

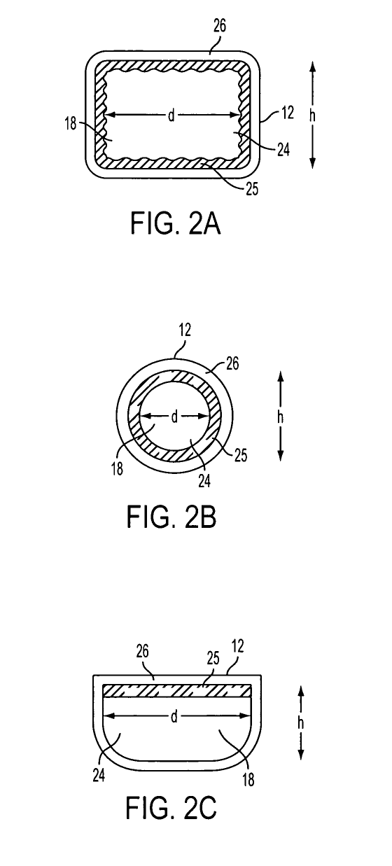

The member 12, shown for example in FIG. 1A, can be configured such that the surfaces of the member 12 define a flow channel 18 having an inlet end 20 and an outlet end 22. At least one of the surfaces 26 defining the channel 18 is transparent so as to make the blood sample in the flow channel visible for purposes of observing the thrombus formation under known microscopy or micro-videography techniques. The transparent surface 26 is preferably made of a non-thrombogenic material, for example, silica materials such as quartz, fused silica, boro silicate glass, plexi-glass or any other glass or plastic surface appropriate for thrombus formation when coated and capable of imaging formation readouts. Member 12 can be made completely of transparent non-thrombogenic material, such as where member 12 is, for example, a micro-capillary tube having a substantially circular cross-section 24. In a preferred embodiment, member 12 is a micro-capillary tube with a central through bore defining flow channel 18. As seen in FIG. 1A, the flow channel 18 defines a longitudinal axis A-A along which the sample of blood can flow. Preferably, flow channel further defines a holding volume of about 20 μl or less, although channel 18 can be configured to hold larger volumes to suit a given assay. Referring to FIGS. 2A-2C, the flow channel 18 further defines a cross-sectional area 24 perpendicular to the longitudinal axis A-A which can be of any geometry. The cross-sectional area 24 is preferably substantially rectangular in shape as seen in FIG. 2A, or alternatively the cross-sectional area 24 can be substantially circular in shape, as is shown in FIG. 2B or substantially semi-circular in shape, as shown in FIG. 2C, although other configurations are possible.

The flow channels 18 of FIGS. 2A-2C define a channel width “d” and height “h”. Preferably, height h is about 200 microns and width d of about 2 mm, more preferably less than about 1.5 mm, even more preferably less than about 1 mm, even more preferably less than about 500 microns and yet even more preferably less than about 400 microns. The channel width d can be constant along longitudinal axis A-A, or alternatively the width d can vary along the longitudinal axis. Varying the width d of flow channel 18 changes the shear rate characteristics of the blood moving through the member 12. This permits a single member 12 to be used to study thrombus formations under varying shear rates of blood flow.

At least one of the surfaces defining the channel 18 can include a coating of thrombogenic material 25 at a concentration so as to facilitate thrombus formation in the channel 18. The thrombogenic material 25 can coat all the surfaces of member 12 defining channel 18, for example, as seen in FIGS. 2A and 2B or alternatively less than all the surfaces may be coated, for example, as seen in FIG. 2C. Preferably, the transparent surface 26 is provided with the thrombogenic material 25. Blood flowing through channel 18 comes in contact with and reacts with the thrombogenic material 25 thereby initiating thrombus formation within the flow channel 18. The thrombogenic material 25 is preferably a collagen, for example, fibrillar collagen type III or fibrillar collagen type I or alternatively, fibrinogen or tissue factor (for example thromborel), although any desired platelet agonists, vascular adhesive proteins for leukocyte recruitment and adhesive matrix with chemoattractant for tumor cell recruitment may be used. The concentration of thrombogenic material 25 can depend on the material used or the extent of thrombus formation sought. For example, collagen can be used at a concentration of about 10 μg per centimeter-squared. In addition, different thrombogenic materials 25 may used in combination in a single member 12 to test anti-thrombotic efficacy under varying conditions. For example, fibrillar collagen type III or I can be used to evaluate the anti-platelet agents directed against GP Ib/IX/V, collagen receptor, GPIIb/IIIa, the ADP receptor in combination with aspirin and hirudin. In another example, fibrinogen can provide information about the GPIIb/IIIa pathway and level of inhibition. In yet another example, thromborel can be used to evaluate anti-thrombotic activity of thrombin receptor antagonists. Alternatively, selectins may be used in place of or along with the thrombogenic materials 25 to study leukocyte recruitment. Alternatively, fibronectin with chemokines may be used to attract circulating tumor cells. To test the anti-thrombotic therapy using different thrombotic agonists, member 12 can be configured to include multiple channels 18 that can run substantially parallel to axis A-A.

Tubing Adapter

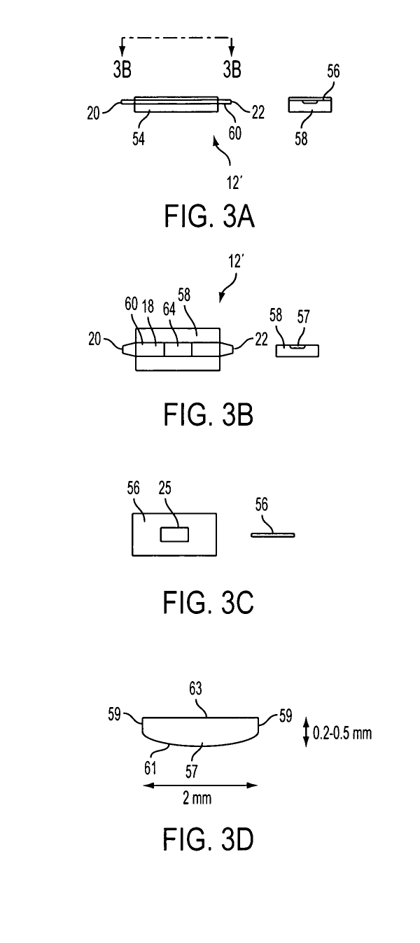

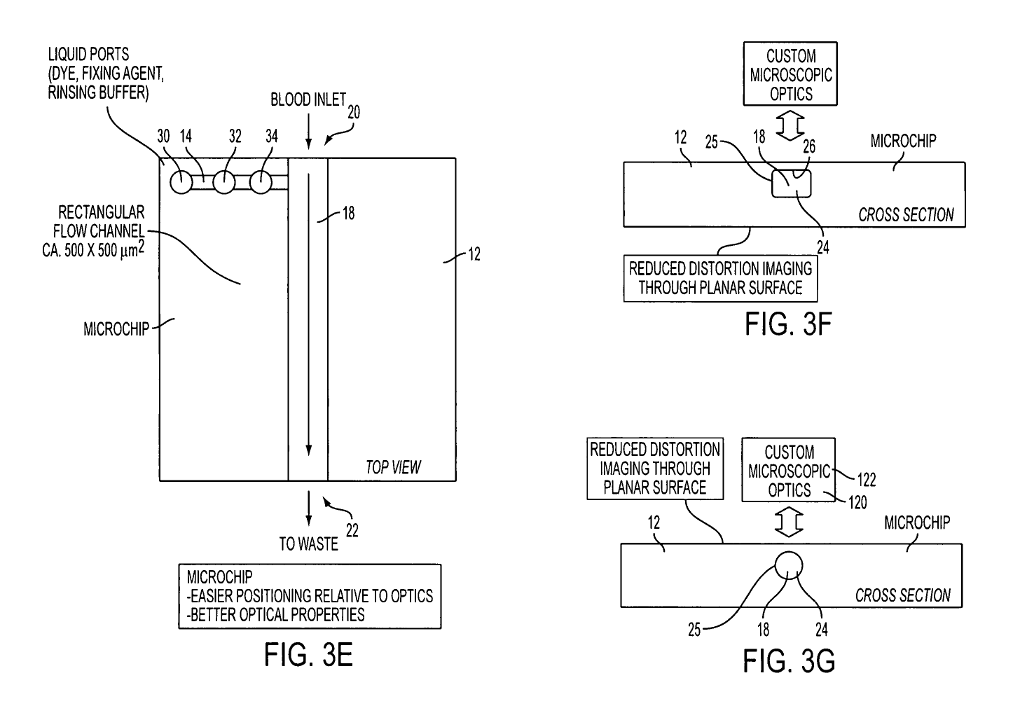

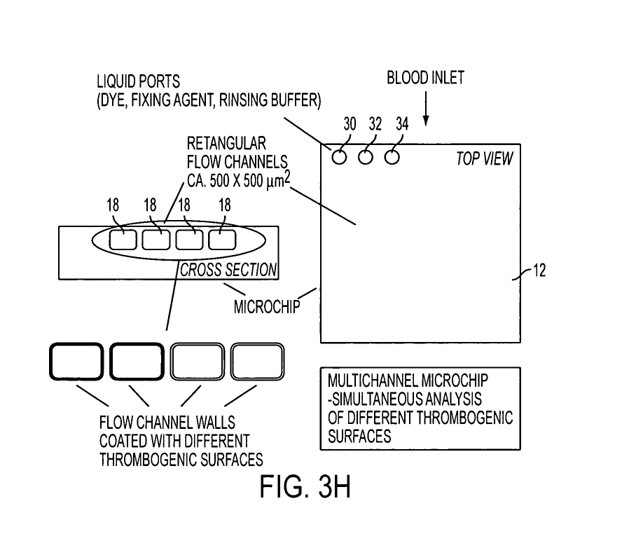

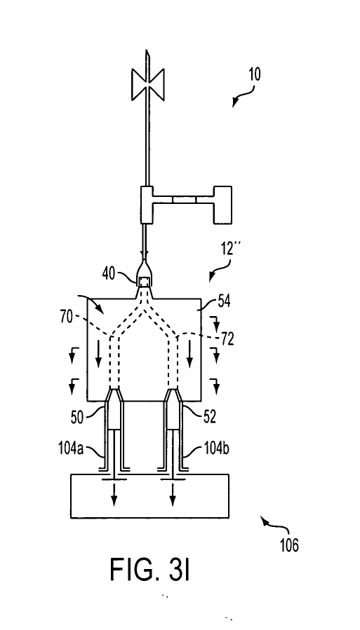

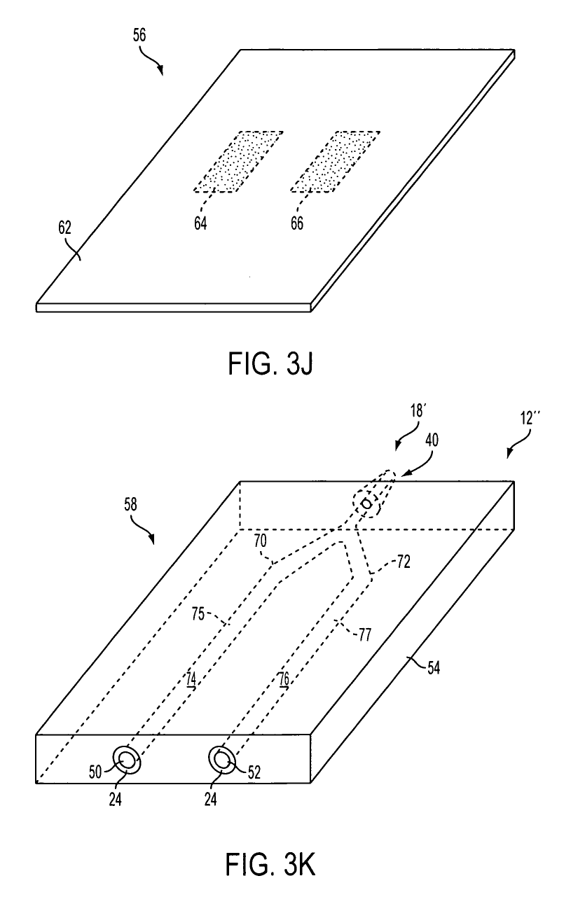

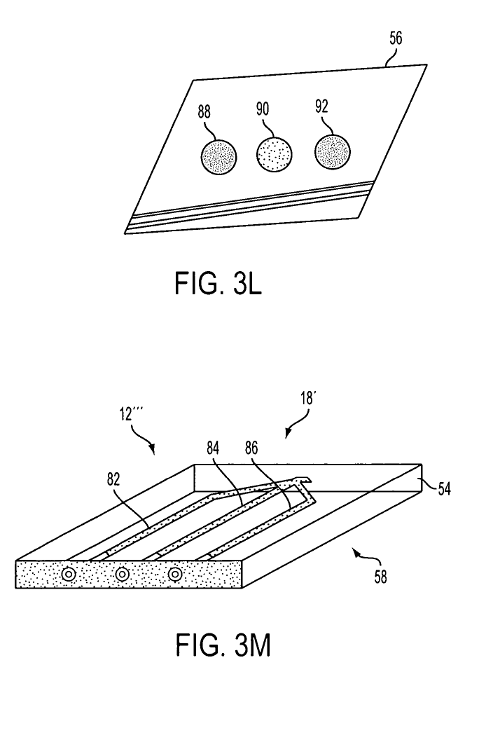

An alternate preferred embodiment of member 12 is shown in FIGS. 3A-3D as member 12′. Member 12′ can include a substantially transparent housing 54 having an upper housing 56 and a lower housing 58. Referring to FIG. 3B, lower housing 58 can be configured to define a channel 57 into which a separable elongated tubing member 60 can be inserted. Channel 57, shown in cross-section in FIG. 3D, is preferably defined by parallel side walls 59 and a substantially arcuate bottom surface 61, typically resulting from micro-fluidic fabrication techniques known in the art. Other volumetric and cross-sectional geometries for channel 57, as previously described with respect to member 12, are possible. Moreover, the cross-sectional geometry can vary along the longitudinal axis, for example transitioning from substantially rectangular to substantially circular along the longitudinal axis or vice versa. The upper housing 56 preferably includes a substantially planar surface that detachably mates with the lower housing 58, as seen in FIGS. 3A & 3C. This planar surface of upper housing 56 defines the preferably planar upper surface 63 of channel 57, as seen in FIG. 3D. The planar upper surface 63 facilitates the imaging of the thrombus formation within tubing member 60 by avoiding any visual distortion due to a curved surface. The channel 57 is preferably about 1-3 mm. wide and ranges in depth from about 0.05 mm. to about 1 mm.

Shown in FIG. 3B, tubing member 60 defines an elongate channel 18 having an inlet end 20 and an outlet end 22 through which a blood sample and imaging agents can flow. Tubing member 60 is preferably configured along its exterior surfaces for insertion into channel 57, thus the geometry of cross-sectional area 24 of tubing member 60, perpendicular to the direction of elongation, can be substantially similar to the cross-sectional geometry of channel 58. Preferably, the cross-sectional area of channel 18 is shaped substantially similar to channel 58. The specific dimensions of channel 18, for example the width, can vary along the direction of elongation. As seen in FIG. 3B, the upper surface of tubing member can include an opening 64. Upper surface 56 can be pre-coated with a thrombogenic material 25 as previously described. Thrombogenic material 25 can be located on upper surface 56 such that upon mating upper surface 56 to lower surface 58, thrombogenic material 25 is inserted into opening 64 and placed in communication with channel 18. Preferably, opening 64 and thrombogenic material 25 are each rectangular shape for complimentary engagement. Thus, when a blood sample is moved through channel 18, the blood reacts with thrombogenic material 25 so as to initiate thrombus formation within channel 18. Alternatively, any surface of tubing member 60 defining channel 18 can be coated with thrombogenic material 25. Lower housing 58 can include multiple channels 57 to hold multiple tubing members 60. Each of the multiple tubing members 60 can be configured such that their total holding volume is preferably smaller than about 20 μl, although larger holding volumes can be provided for a given application. Each tubing member 60 can be variably coated with thrombogenic material 25, as is required for performing the desired assay. Moreover each channel 18 of tubing member 60 can be variably dimensioned with respect to one another for multiple shear measurements.

Inlet and outlet ends 20, 22 of tube member 12, 12′ can be dimensioned and configured to connect to fluid handling elements of the fluid handling assembly 14, for example, outlet end 22 can be connected to tubing, for example, silastic tubing, that is connected to a syringe pump or alternatively, a collection vessel. Preferably, tubing member 60 and housing 54 are made of non-thrombogenic material and are compatible, i.e. transparent and non-fluorescent, for use in light microscopy or videography using fluorescence or K \overline{h}ler Illumination to facilitate the imaging of thrombus formation in the channel 18. Assembled housing 54 with tubular member 60 can serve as a disposable, perfusion chamber, pre-coated with thrombogenic material 25, for use in the instrument 10 thereby possibly enabling ease of operation of instrument 10 and higher reproducibility in blood assay studies. This flexibility in using tubular member 60 can increase the ease and productivity in performing assays for a large sample study. Preferably, assembled housing 54 and tubular member 60 can be provided in a disposable kit form (not shown) which can further include tubing connected to a needle to pierce a vacutainer collection vessel or other collection means, and a tubing and syringe assembly for insertion into a separate syringe pump.

Microchip Based Device

In yet another embodiment of member 12, shown in FIG. 3E, member 12 can be constructed from a microchip in manner known to one of ordinary skill in the art of microfluidic applications. The microchip member 12 can be constructed from a substantially planar glass (or any transparent material) microchip having a surface 26 defining a channel 18 at least partially coated with a thrombogenic material 25. A sample of blood can be moved through channel 18, which defines a preferably substantially rectangular cross-section area 24 as seen in FIG. 3F. Alternatively, the cross-sectional area 24 can be substantially circular, as shown in FIG. 3G, or another geometry. Moreover, the cross-sectional geometry can vary along the longitudinal axis, for example transitioning from substantially rectangular to substantially circular along the longitudinal axis or vice versa. The construction of member 12 as a microchip facilitates implementation of flow channel 18 with cross-sectional area 24 having varying geometries. The rectangular cross-sectional area 24 minimizes the optical distortion in imaging of the channel 18 due to the planar surfaces defining the channel 18. Where channel 18 defines a circular cross-sectional area 24, any distortion due to the arcuate surface 26 defining the channel 18 is minimized by the external planar surfaces of the microchip member 24.

Given the viscosity of the blood due to the cellular components in the blood, flow characteristics of the blood sample can be varied by varying the width or diameter of the vessel or channel through which the blood flows in the direction of flow. Therefore, for hemodynamic reasons, the channel 18 of microchip member 12 can be about 2 mm, more preferably less than about 1.5 mm, even more preferably less than about 1 mm, and yet even more preferably about 500 microns wide, which is larger than typical channel dimensions in microfluidic applications known in the art. More preferably however, the channel 18 of microchip member is less than about 400 microns. Microchip member 12 can also be configured to include as many channels 18, and as variably coated with thrombogenic, procoagulant or pro-inflammatory materials 25, as is required for performing the desired assay. The channels 18 can be variably dimensioned with respect to one another so as to permit multiple shear measurements. Preferably microchip member 12 is configured such that its total holding volume is preferably smaller than about 20 μl, although larger holding volumes can be provided for a given application. Shown in FIG. 3H is microchip member 12 having multiple channels 18.

Like the member 12′, microchip member 12 can offer a pre-coated and disposable chamber in which to conduct and hold a blood sample assay. An additional advantage in configuring instrument 10 as a microchip based system, when performing fixed end point measurement imaging of thrombus formation, can be the elimination of the need to image the thrombus formation immediately following a single assay. The blood sample assays can be performed separately in batch processes using instrument 10. With the thrombus formations fixed and stained within the microchip members 12, the imaging of the microchip members 12 can be performed at a later time also in a separate batch process.

In FIG. 1F, imaging assembly 15 is preferably a part of instrument 10 and utilizes socket 38 as a stage for imaging member 12. Alternatively, imaging assembly 15 can be independent of instrument 10 and have a socket similarly configured to socket 38 for securing and orienting member 12 with respect to the microscopy optics for imaging. In this alternative embodiment, previously assayed members 12 can also be imaged in a batch process. Batch mode end point reading, for example, can be preferable for drug discovery to report result alternative applications compared with acute/chronic coronary settings.

Planar Housing

Shown in FIGS. 31-3M is yet another alternative embodiment of member 12 in the form of a perfusion chamber member 12″. Perfusion chamber member 12″, shown in perspective view in FIGS. 3J and 3K is preferably a generally flat housing 54. Housing 54 can be formed of two mating portions: upper housing 56 and lower housing 58. Lower and Upper Housing 56, 58 portions may be joined so as to form a fluid tight seal therebetween, for example by heat sealing, joint adhesive sealing or any other techniques known to one of ordinary skill in the art for fluid tight sealing.

Lower housing 58 can be a generally flat, preferably rectangular housing having a defining flow channel system 18′ substantially along longitudinal axis A-A through which a blood sample can be moved. Preferably, channel system 18′ includes a single inlet channel 40 which splits into two substantially parallel flow channels 70, 72 which terminate respectively at outlets 50, 52 coterminous with the body 68. Alternatively, flow channels 70, 72 can be configured with independent inlets. Flow channels 40, 70, and 72 define cross-sectional area 24 which is preferably circular, although other cross-sectional geometries are possible. Moreover, the cross-sectional geometry can vary along the longitudinal axis, for example transitioning from substantially rectangular to substantially circular along the longitudinal axis or vice versa. Flow channels 40, 70 and 72 each define a diameter d′ which may vary along the channel 18′ in the direction of axis A-A. Alternatively, diameter d′ may be constant along the axis A-A. In addition, the dimensions or geometry of the cross-sectional area 24 of flow channels 70 can be different than the cross-sectional area of flow channel 72. Flow channels 70, 72 can be configured such that their total holding volume is preferably smaller than about 20 μl, although larger holding volumes can be provided for a given application.

Upper housing 56 can be a substantially flat plate defining an interior surface 62 in communication with the channel system 18′. Thrombogenic material 25, as previously described, may be coated along a portion of the interior surface 62 for facilitating thrombus formation in the channel system 18′ when the blood sample is moved therethrough. More specifically and preferably, the thrombogenic materials 25 are applied along a portion interior surface 62 in communication with channels 70, 72 to facilitate thrombus formation therein. The thrombogenic materials 25 used in, for example, flow channel 70 can be different than the thrombogenic material 25 used in flow channel 72 to observe varying anti-thrombotic reactions. For example, the thrombogenic material 25 in flow channel 70 may be of a different type than the thrombogenic material 25 in flow channel 72, or alternatively, the thrombogenic material 25 in channel 70 may vary in concentration from the thrombogenic material used in channel 72. Upper housing 56 is preferably made from a transparent non-thrombogenic material in order to facilitate the micro-videography or microscopy imaging of the thrombus formations in flow channels 70, 72.

The member 12″ shown in FIG. 3K includes two substantially parallel flow channels 70 and 72. In an alternative embodiment, as shown in FIGS. 3L and 3M, the perfusion member 12′″ can include at least three flow channels 82, 84 and 86. Each flow channel 82, 84 and 86 can be separately configured in a manner similarly described with respect to flow channels 70 and 72. In addition, each channel 82, 84, and 86 can have a surface 80, 90, 92 in communication with the channel 82, 84, and 86 that is coated with varying thrombogenic materials 25. Alternatively, member 12′″ may be configured so as to define as many flow channels in the system of channels 18″ as is needed for a blood therapy study.

Referring back to FIGS. 1C and 1F, instrument 10, 10′ can include a receiver member or socket 38 configured for holding and orienting member 12 in a specific manner with respect to the remaining components of instrument 10. More specifically, socket 38 can be configured so as to properly secure and orient member 12 for proper imaging of the thrombus formations within channel 18. Socket 38 can be a holder 39 including a chamber 37 for housing the member 12 and tubing. For example, shown in FIG. 1G is a preferred embodiment of a holder 39 having a chamber 37 for housing the member 12. Socket 38 can be further configured to hold piping, for example, a single silastic tubing from a blood sample reservoir to the member 12 and another silastic tubing from the member 12 to the pump (not shown).

In another example, socket 38 can have a connection fitting that complementarily mates with the connection fitting of micro-capillary tube member 12 such that the transparent surface 26 is oriented with respect to imaging assembly 15 in order to image the thrombus formation inside channel 18 with the appropriate resolution and magnification. For example, socket 38 can include a telescopic stage that could be operated to bring the channel 18 into focus with respect to imaging assembly 15.

Socket 38 can be further configured so as to properly secure and orient member 12 for a liquid tight connection to the blood sample source, imaging agent source and fluid handling assembly 14. For example, socket 38 can include fluid handling fittings and elements known to one of ordinary skill in the art so as to, for example, properly deliver a blood sample or imaging agent flow channel 18. More specifically, socket 38 can include, for example, a quick disconnect coupling to permit easy and quick insertion and disconnection of member 12 from a fluid handling element of the fluid handling assembly 14, for example, a pump. In another example where member 12 can be embodied as a microchip member 12, instrument 10 can include a socket 38 for complimentary “snap-in” arrangement with microchip member 12, thus facilitating easy change-out of the microchip member 12 and set up of instrument 10 for multiple assays.

Fluid Handling Assembly

Referring again to the schematics of FIGS. 1A and 1D, instrument 10, 10′ includes fluid handling assembly 14 which can have one portion 14a for handling delivery of a blood sample to member 12 and moving the blood sample through the channel 18. Fluid handling assembly 14 can have another portion 14b for handling delivery of other liquids, (not shown in FIG. 1A) for example, image enhancing agents to channel 18.

Fluid handling portion 14a preferably moves a blood sample through channel 18 of member 12 by vacuum pressure. As seen in FIGS. 1C and 1D, fluid handling portion 14a can be single tubing, for example silastic tubing connected to inlet and outlet ends 20, 22 of member 12 to connect to the reservoir sample of blood and the syringe pump. For example, and as seen in FIG. 3I, flow channels 70 and 72 can be connected at their outlet ends 50, 52 to separate syringes 104a, 104b respectively. Syringes 104a, 104b can be conventional type syringes including pistons for creating a vacuum. Syringes 104a, 104b can be connected to a pump 106 to operate the pistons of syringes 104a, 104b. Pump 106 can be a commercially available peristaltic pump, for example, a Harvard Apparatus Pump. Additionally, fluid handling portion 14b can include tubing, valves and connection fittings to draw blood from a sample source and deposit the sample to a waste vessel upon exit from member 12. Preferably, all tubing, connections and fluid handling elements are made of non-thrombogenic material.

A blood sample can be moved through channel 18 of member 12 at a user selected shear rate which is expressed in units of per second (s⁻¹). For example, the blood sample can be moved through channel 18 at a shear rate that mimics the human arterial shear rate estimated to be about 600-800 per second, shear rates found in moderate stenosed arteries (1500-10000/sec) or alternatively mimic the human venous shear rate of about 50-200 per second. In this manner, a blood assay using instrument 10 can model thrombus formation in a vein or artery. In addition, the shear rate of flow through member 12 can be selected so as to account for stenosis, where a moderately stenosed artery can result in a shear rate of about 1,500 per second, and a severely stenosed artery can result in a shear rate of about 6000 per second.

Shear rate can be a function of both the volumetric flow rate “Q” and the cross-sectional geometry of the channel through which a fluid flows. For example, where channel 18 defines a substantially rectangular cross-sectional area 24 having a width “a” and a height “b,” the shear rate at the wall shown in equation (1):
γ_{at wall}=1.03*Q/(a*b²) (1)

Where cross-sectional area 24 is substantially circular having a radius “r” the shear rate is found by the equation (2):
γ_{at wall}=4*Q/(π*r³) (2)

In order to regulate or adjust the shear rate to mimic blood flow through veins or arteries, the flow rate can be adjusted by accordingly changing the flow rate of the pump or otherwise changing the geometry of the channel 18. For example, as previously described, member 12 can be configured so as to vary the width d of channel 18 in the direction of flow along the longitudinal axis A-A.

Fluid handling portion 14b can be configured to deliver various imaging enhancing agents to facilitate proper imaging of the thrombus formation. For example, in kinematic imaging of the thrombus formation in channel 12, preferably a fluorescent label, for example, Rhodamine 6G in saline, is added directly to the sample of blood so as to reach a concentration of about 1-10 micrograms/ml. Alternatively, the blood can be fluoresced using Mepacrine at a concentration of about 0.2 mg/ml as a dye. The dye can be added to the whole sample prior to or during perfusion. In addition, a blood sample to be kinematically imaged is preferably slightly anti-coagulated. The fluid handling assembly 14 can be configured to deliver a small amount of anti-coagulant, for example, Ppack, citrate, heparin, EDTA, a factor Xa inhibitor or any other anti-coagulant known in the art, to the blood sample prior to perfusion.

Alternatively, the thrombogenic surface or the material coated onto the thrombogenic surface can be fluorescently labeled. Quenching of the fluorescent surface due to platelet deposition or any other cells becomes the read-out of the thrombotic process for example.

Fluid handling portion 14b can be configured for facilitating fixed end point measurement imaging or other alternative imaging techniques to micro-videography. For example, after fluid handling portion 14a moves or perfuses a blood sample through channel 18 so as to initiate thrombus formation, fluid handling portion 14b can deliver image enhancing agents to fix and stain the thrombus formation within the channel 18 in accordance with, for example, light microscopy techniques know to one of ordinary skill in the art. Imaging enhancing agents can include: (i) a rinsing buffer; (ii) a fixing solution of either PBS or glutaraldehyde 2.5% or PBS, PFA 4%; and (iii) a stain solution, i.e. toluidin blue solution form Becton Microscopy Science. Fluid handling assembly 14 can include the requisite tubing, piping and handling elements needed for delivery of the image enhancing agents to the channel 18. In addition, a control system can be interfaced with fluid handling portion 14b to automate the sequencing and metering control of the delivery of the image enhancing agents.

Fluid handling assembly 14 can include one or more fluid control elements 100, for example, a valve that controls the flow of the blood sample into the blood sample channel 18. Any piping components, fitting and/or elements located between the blood sample reservoir and the tubing member 12 is preferably constructed from non-thrombogenic material and preferably constructed so as not to disturb the laminar flow of the blood sample through member 12 in order to avoid activating the platelets. These fluid control elements 100 can be configured for automatic operation by a properly interfaced control system.

In the case of where member 12 is specifically embodied as the microchip member 12 of FIGS. 3E and 3H described above, the microchip member 12 can include fluid handling portion 14b that delivers the image enhancing agents, i.e. dye, fixing agent, rinsing buffer, etc. More specifically, microchip member 12 can include liquid ports 30, 32, and 34 of fluid handling assembly 14. Each of liquid ports 30, 32 and 34 can be configured for delivery of any one of the image enhancing agents. The liquid ports 30, 32 and 34 can be configured so as to deliver the image enhancing agents directly into the channel 18. Alternatively, the microchip member 12 can include only a single liquid port, for example, liquid port 30 to deliver all the necessary image enhancing agents.

Imaging Assembly

Imaging assembly 15 is preferably configured for kinematic imaging of the thrombus formation or recruitment of any circulating blood cells in channel 18 of member 12 using light microscopy and/or micro-videography techniques involving fluorescence illumination as is known in the art. Imaging assembly 15 of instrument 10 includes fluorescence excitation optics, to imaging a time-lapse video or motion picture of thrombus formation.

Referring to FIGS. 1A and 1B, imaging assembly 15 of instrument 10 includes fluorescence excitation optics, for example, a light source 122 and a microscope 120 interfaced with a camera 124 for imaging a time-lapse video or movie of thrombus formation. Preferably, camera 124 is a CCD camera with microscopic zoom capability to eliminate the need for a separate microscope. Camera 124 can be, for example, a Nikon DXM1200 digital camera. Preferably, camera 124 is a digital monochrome video camera having 8-bit, integration times ca. 500 ms, IEEE 1394 interface wherein images are acquired at 1-3 Hz. Microscope 120 preferably has a magnification of 20× and includes excitation and emission filters and a dichroic mirror. Light source 122 is preferably an LED, and more preferably, light source 122 can be a high power green LED having a preferred wavelength of about 530 nm with a narrow spectral distribution and low power consumption. Alternatively, multiple fluorescent measurements, for example using red or blue LED can be enabled to perform complex assays in which a computer controlled analyzer can support the wavelength, exposure and flow parameters of the experiment including saving the data for analysis.

Shown in FIG. 1C is an arrangement of instrument 10 showing relative positions of the member 12, fluid handling assembly 14, and imaging assembly 15 in an enclosure 17. The imaging assembly 15 is disposed proximate the member 12. Specifically, member 12, light source 122 and the objective of microscope 120 can be disposed relative to one another such that the light source 122 can illuminate the channel 18 and the microscope 120 can magnify and resolve the thrombus formation in channel 18 as the thrombus formation develops. The microscope 120 can be disposed relative to the transparent surface 26 of member 12 in order to focus on the thrombus formation in channel 18. The enclosure 17 is configured to substantially house the instrument 10 and also filter or block out surrounding room lighting so as not to interfere with the fluorescence imaging of the thrombus formation.

During perfusion of the fluorescent labeled blood sample through member 12, the blood sample reacts with the thrombogenic material 25 to begin thrombus formation within channel 18. Fluorescent platelets adhere to the coated surface, thus initiating aggregation of individual platelets to form the thrombi. The imaging assembly 15 repeatedly images the thrombus formation developing in channel 18. The thrombus formation adheres and aggregates along the surfaces of channel 18 coated with thrombogenic material 25. The fluorescent labeled platelets appear in the field of view of the microscope 120. The illumination from the light source 122 passing through member 12 visually enhances the view of the fluoresced thrombus formation. The lenses of the microscope 120 resolve and magnify the image of the thrombus formation with sufficient contrast so as to enable image capture and analysis of the formation.

The preferred camera 124 of imaging assembly 15 captures the fluoresced image of the evolving thrombus formation as digital image data, a sample of which is shown in FIG. 1H. The frame rate of the camera 124 of imaging assembly 15 is preferably about 2 frames per second to capture the thrombus formation as a time-lapse motion picture. Other frame rates are possible but may require larger image data file sizes and hardware. The digital data image can be stored to read/write digital medium 137 in, for example, a hard drive of a computer or alternatively a networked data storage device.

Imaging assembly 15 can alternatively and optionally include a non-imaging photodetector 127, for example, a photodiode or photomultiplier. The photodetector 127 produces an electrical signal response to light emitted from the fluoresced thrombus formation. The electrical signal can be read, processed, and correlated by computer 136 to quantify the temporal evolution of thrombus formation and any other characteristics of the thrombus formation. The photodetector 127 can be used to provide a more sensitive, better signal to noise measurement of thrombus formation in parallel with the time-lapse video.

In addition, instrument 10 can be configured for performing both kinematic time lapse imaging of the thrombus formation and alternate fixed end point measurement imaging. In order to perform fixed end point measurement imaging, instrument 10 can be configured in a manner as described below with respect to instrument 10′.

Alternatively, imaging assembly 15 can be configured for fixed end point imaging of the thrombus formation in channel 18 of member 12 using light microscopy techniques and optics involving K \overline{h}ler illumination as is known in the art. In contrast to the kinetic imaging of thrombus formation, fixed end point imaging captures a point in time image, the “end point” of the thrombus formation after perfusion of the blood sample through the member 12 and after the thrombus formation has been fixed and stained in the channel 18. Shown in FIG. 1D, is a schematic view of instrument 10′ and imaging assembly 15 relative to the member 12. Preferably, imaging assembly 15 includes a light microscope 120 and a light source 122. Light source 122 is preferably an LED and more preferably, light source 122 can be a high power green LED.

Shown in FIG. 1F is an arrangement of instrument 10′ showing relative positions of the member 12, fluid handling assembly 14, and imaging assembly 15 in an enclosure 17. Like instrument 10, the imaging assembly 15 in instrument 10′ is disposed proximate the member 12. Member 12, light source 122 and the objective of microscope 120 can be disposed relative to one another such that the light source 122 can illuminate the channel 18 and the microscope 120 can magnify and resolve the thrombus formation in channel 18 where the thrombus formation had been previously fixed and stained within the channel 18 by the image enhancing agents as previously described. In K \overline{h}ler illumination, the light source 122 illuminates the fixed and stained thrombus formation. Light beams passing through the thrombus formation are refracted and captured in the object lens of the microscope 120. The lenses of the microscope 120 resolve and magnify the image of the thrombus formation with sufficient contrast so as to enable analysis of the formation.

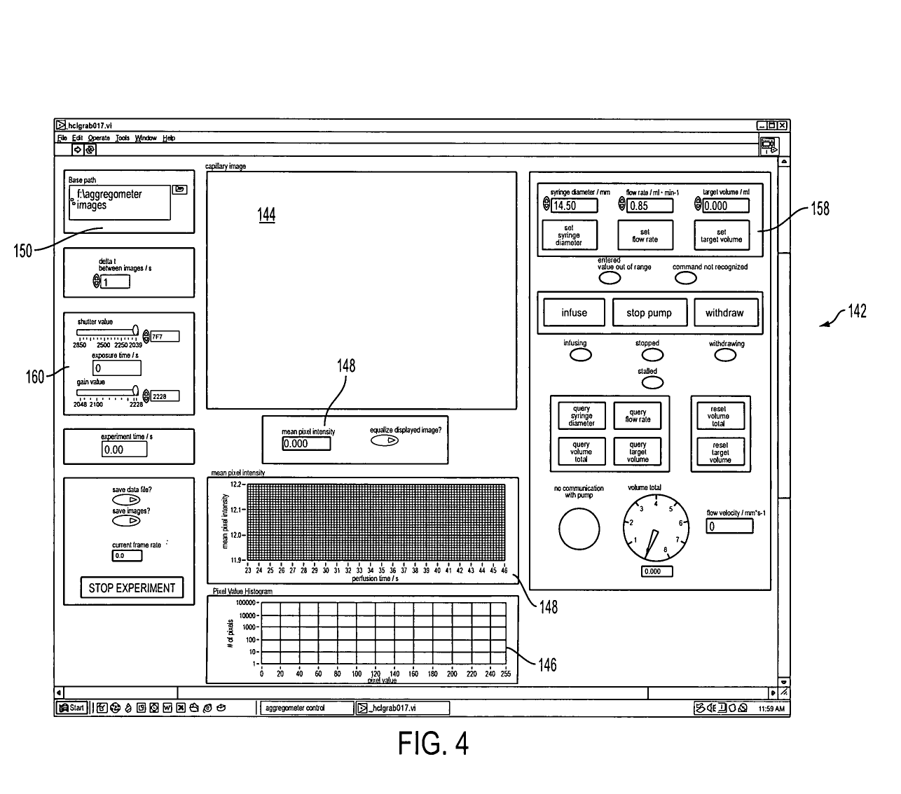

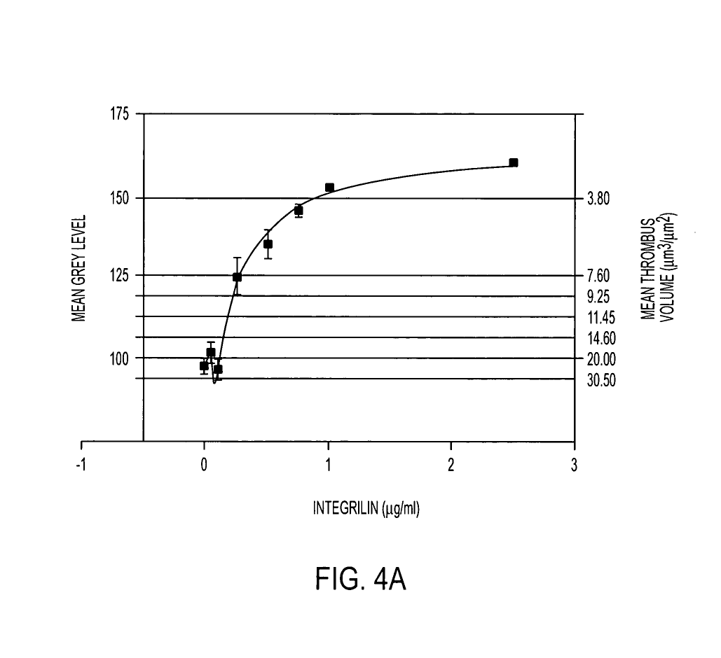

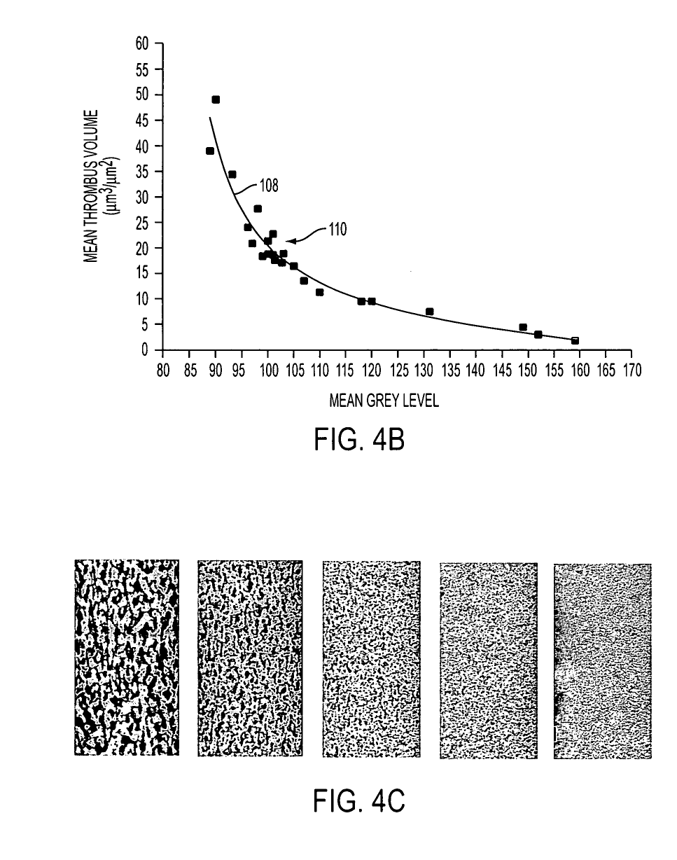

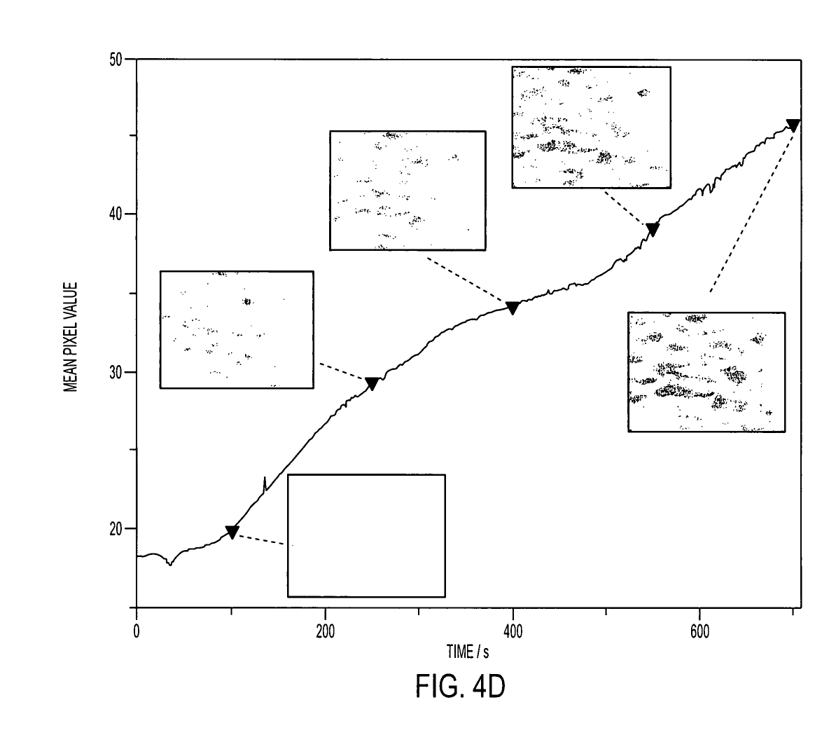

In order to capture the image of the thrombus formation in the channel 18, imaging assembly 15 can also include a camera 124, shown schematically in FIG. 1D. More specifically, imaging assembly 15 can include a CCD camera 124 for converting the light image of the thrombus formation to a fixed digital data image, a sample of which is shown in FIG. 4C. The digital data image can be stored to read/write digital medium 137 in, for example, a hard drive of a computer or alternatively a networked data storage device. As in instrument 10, camera 124 of instrument 10′ can preferably include a microscopic zoom lens to eliminate the need for the separate microscope 120. Alternatively, camera 124 can be interfaced with microscope 120 to digitally capture the image of the thrombus formation.

Alternative light contrasting techniques can be employed to image the thrombus formation as are known to one of ordinary skill in the art of microscopy. Such techniques include: (i) Oblique illumination; (ii) polarization; (iii) phase contrast; (iv) acoustic microscopy; and (v) differential interference contrast.

The Analyzer

The digital image data of thrombus formation captured by digital camera 124 in either embodiment of instrument 10, can be stored, displayed and printed or otherwise processed to quantify certain aspects of the thrombus formation, for example, the volume of thrombus formation. Instrument 10 can include an analyzer 16 having a processor 132 including an interface 134 for receiving and reading digital image and non-image data of the thrombus formation.

Processor 132 can preferably be a computer 136 having serial connection to digital camera 124 to receive the digital image data. More preferably the camera 124 is connected to computer 136 by a firewire connection for rapid digital image data transfer. Alternatively, computer 136 can have a disk drive as is known in the art for receiving and reading the digital image data stored to a portable read/write recording medium 125 of the camera 124. Processor 132 can convert the digital image data to pixel data in a manner known to one of ordinary skill in the art. Pixel data can include, for example, pixel color or pixel intensity. Processor 132 can further use the pixel data using at least one algorithm 138 to correlate and/or quantify an aspect of the thrombus formation, i.e., the volume of thrombus formation.

Preferably, computer 136 can include executable software or computer program 140 capable of running the algorithm 138 to read the digital image data and convert it to pixel data to calculate and display the quantifiable aspects of thrombus formation. The computer program 140 can be written and customized using known data acquisition software, for example, LabView software. The pixel data determined by program 140 can be correlated to thrombus formation in accordance with user selected needs. For example, pixel data indicating dark colors may be correlated to indicate the presence of thrombus formation; therefore, large clusters of dark colored pixel data indicate the presence of a high concentration of thrombus formation. Alternatively, program 140 may be configured such that a cluster of light colored pixel data indicates the presence of thrombus formation. The pixel data can be used to display the image of the thrombus formation to a display device, for example, a computer monitor or for printout by a computer printer. Shown in FIG. 4D are graphically shown sample still images of evolving thrombus formation shown by temporal change in mean pixel value taken with the imaging assembly 15 of the instrument 10 using kinetic imaging.

The computer program 140 can include a routine to generate a user interface 142 having a data display that can be displayed on a computer monitor to report measured and correlated data concerning the thrombus formation. For example, as seen in the screen shot FIG. 4, shown is a user interface 142 generated by program 140 for displaying the thrombus formation and the calculated parameters of the thrombus formation correlated with the digital image data. Interface 142 can include a thrombus formation display 144 showing the thrombus formation within a portion of the channel 18 of member 12, a pixel value histogram 146, a graph 148 showing the time rate of change in mean pixel intensity, and a mean pixel intensity read out 148 displaying the calculated mean pixel intensity. The program 140 can be further configured to provide read outs of the calculated volume of thrombus formation or the time rate of change in volume of thrombus formation (not shown).

As previously described, instrument 10 and imaging assembly 15 can include a non-imaging fluorescence photodetector 127, for example, a photodiode or photomultiplier which for converting the fluorescence intensity of the platelets aggregated in the field of view to an electrical signal or other non-imaging data. In instrument 10, a computer 136 is preferably provided having software program 140 including algorithm 180 which can process non-imaging data received from the photodetector 127. The software program 140 can be for example, LabView software including an analog to digital converter for reading the electrical signal. The software program 140 can integrate the captured fluorescence intensity over the entire field of view to give a thrombus formation curve 190 as is schematically shown in FIG. 1A. The curve 190 and its data can be further processed by program 140 to provide a temporal evolution of the volume of thrombus formation in the channel 18 and/or other quantifiable characteristics of thrombus formation.

Shown in FIGS. 1A and 1C is the analyzer 16 of FIG. 1 being a computer 136 preferably disposed proximate the imagining assembly 15 to permit immediate correlation of either (i) the digital image data or (ii) the non-imaging data as it relates to the thrombus formation. The data can be stored to the local read/write memory or hard drive of the computer 136. However, alternatively, analyzer 16 can be completely separated from the imaging assembly 15 and instrument 10. In one embodiment, analyzer 10 can include a stand alone computer 136 including a software or computer program 140 with at least one algorithm 138 as previously described. Bundled detector or digital image data of blood assays can be delivered to computer 136 for analysis. For example, bundled digital data image files can be stored on a read/write recording medium 125 of imaging assembly 15 in one location and downloaded for analysis on the computer 136 in another location and stored to a data storage device or medium 137 in the same or different location. The digital image data files can be read from the portable read/write recording medium 125 using a disc drive as is known in the art. Alternatively, the digital image data files can be stored on a server 137, for example, on a local or wide area network, for example, on an intranet or the Internet. Shown in the screen snapshot of FIG. 4, interface 142 includes a user selector control 150 that permits a user to browse local or network drives for either saving digital data image files for later analysis or accessing previously saved digital image data files for immediate analysis. Permitting bundled data files concerning the thrombus formation to be stored for later analysis permits for high volume blood assays and imaging to be performed without having to run the thrombus formation analysis in sequence with the imaging.

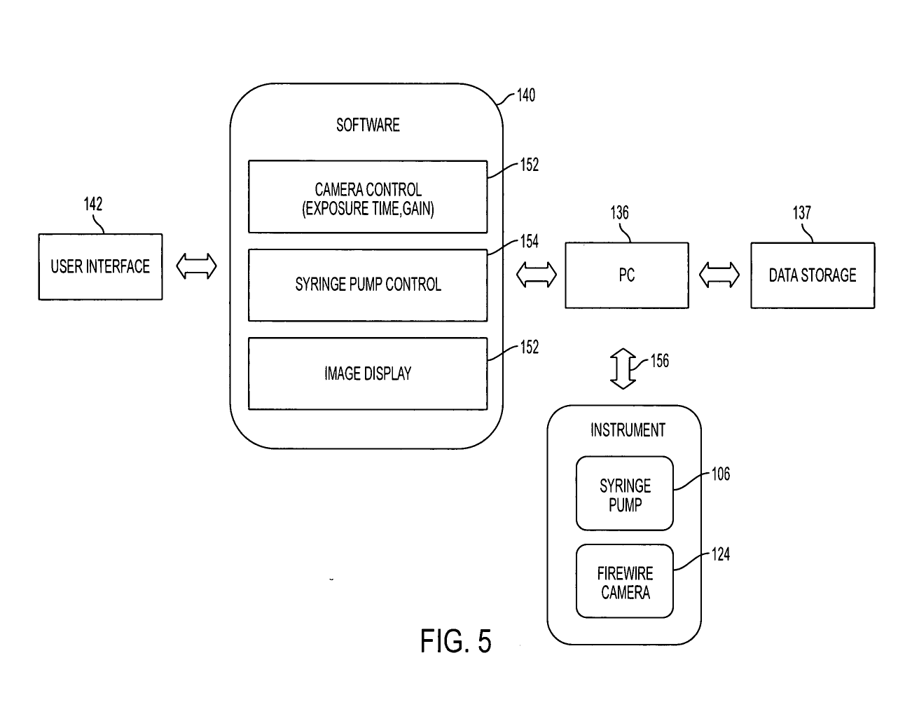

Program 140 may include additional algorithms to control other features of instrument 10, 10′. Referring now to FIG. 5, software program 140 can preferably include an imaging control algorithm 152 for controlling the imaging assembly 15 and a fluid control algorithm 154 for controlling the delivery of fluids to the channel 18 of member 12 or directly to the blood sample. For example, the imaging control algorithm 152 can be configured to control the exposure times and setting of camera 124 of imaging assembly 15, wherein the computer 136 and the camera 124 preferably communicate via a firewire interface. Alternatively, algorithm 152 can be configured to control any of the previously described operations of the imaging assembly 15.

In another example, the fluid control algorithm 154 can be configured to control the off/on function or the variable flow rate of pump 106. Moreover, in assays utilizing multiple channel 18 embodiments of member 12, the fluid control algorithm 154 can be configured to vary the flow parameters from channel to channel. In addition, algorithm 154 can be configured to control, for example, the sequencing or off/on delivery of the image enhancing agents used in the fluid handling assembly 14. Fluid handling assembly 14 and imaging assembly 15 can be controlled by using an appropriate interface between the computer 136 executing program 140 and its algorithms 152, 154 and the equipment to be controlled. Shown schematically in FIG. 5 is the interface 156 between computer 136 and the pump 106 and camera 124. Although FIG. 5 shows algorithms 152 and 156 as part of the same program 140 used in the analysis of digital image data files, it is possible for algorithms 152 and 156 to be configured to operate independent of one another and the analysis program 140. Independent arrangement of programs and their algorithms may be particularly necessary when, for example, the analyzer 16 is independent of the remainder of instrument 10.

The delivery of the image enhancing agents, in terms of either volumetric or sequential control, can be automated by a fluid control algorithm or system 154 (shown in FIG. 5) interfaced with liquid handling assembly 14. For example, referring again to FIGS. 3E and 3H, microchip member 12 can include the requisite fluid and electrical/electronic interfaces (not shown) known to one of ordinary skill in the art for connection to the blood sample source, imaging agents source, fluid handling assembly 14, or fluid control algorithm 154. It is to be understood that liquid ports 30, 32 and 34, fluid handling assembly 14 and fluid control algorithm 154 can be configured so as to deliver any agent needed for the purpose of the blood assay.

It may be desirable to configure algorithms 152, 154 so as to permit a user to select specific values for process parameters for use in, for example, the automatic control of the pump 106 or camera 124. Shown in the screenshot of FIG. 4 is user interface 142 through which a user can interface with control algorithms 152, 154. User interface 142 can include user controls 158, 160 for interfacing with the pump 106 and the camera 124 respectively. Controls 158 and 160 can include one or more numerical entry fields and setting buttons. Control 158 can be configured to permit a user to set flow characteristics of the pump 106 so as to a experience a target shear rate in the channel 18 when moving the blood therethrough. Flow characteristics can include the flow rate of the pump 106 or the chamber diameters of the syringes 104. Controls 160 can be configured to permit a user to set, for example, the exposure time, gain and shutter value of camera 124 in order to produce the desired resolution of the thrombus formation image.

### III. THE METHOD

Instrument 10 can be operated in the following manner. Member 12 is prepared by providing thrombogenic material 25 on at least one of the transparent surfaces 26 defining channel 18 in order to initiate and promote thrombus formation therein. Depending on the configuration of member 12, as described above, member 12 can be pre-coated with the thrombogenic material 25, for example, on the upper surface 56 of the member 12′ having an adjusting tube member 60. Alternatively, member 12 can be manually coated with the thrombogenic material 25 prior to running the assay, for example, using micro-capillary tube member 12. Member 12 is then assembled based upon its construction, as previously described, and inserted into the socket 38 of instrument 10 for secure holding and orientation relative to the remaining components of the instrument 10. Any necessary tubing, for example silastic tubing, is provided to connect the blood sample with the member 12 and the fluid handling assembly 14. Additionally, a rinsing buffer of, for example, a saline mixture can also be run through the tubing of instrument 10 to avoid air from developing in the piping system.

In a preferred method in which the thrombus formation is imaged using kinetic or time lapse imaging of the formation, the blood sample is preferably labeled with a fluorescent agent and slightly anti-coagulated with a small amount of anti-coagulant, for example, heparin, Ppack, citrate, EDTA, factor Xa inhibitor or any other anti-coagulant known in the art, while in the reservoir and prior to perfusion through member 12. Preferably, fluid handling assembly 14 uses vacuum pressure to draw the fluorescent blood sample through the channel 18 of member 12. Specifically, fluid handling assembly 14 includes a syringe pump 106 having a known flow rate so as to move the sample of blood through the channel 18 having a cross-sectional area 24 of preferably known dimensions at a desired shear rate. More preferably, instrument 10 includes a computer 136 running a software program 140 including algorithm 154 in conjunction with user interface 142, as shown in FIG. 4, having controls 158. A user can use controls 158 to set the flow rate of fluid handling assembly 14 or pump 106 to move the blood sample at a desired shear rate. The fluid handling assembly 14 operates to draw the blood through channel 18 of member 12 for a period of time sufficient for the blood to react with the thrombogenic material in channel 18 and initiate thrombus formation in the channel 18. The period of time the fluid handling assembly 14 operates to move the blood sample through the channel 18 can be controlled by algorithm 152 and the user settings input into controls 158 of user interface 142.

Referring back to FIGS. 1A and 1B, during perfusion of the blood sample through the member 12 and as previously described, the imaging assembly 15 repeatedly images the channel 18 at defined intervals to capture the evolving thrombus formation. Member 12 is preferably maintained in socket 38 of instrument 10 for microscopy imaging by the imaging assembly 15 in accordance with the microscopy techniques described above. Preferably, computer 136 having software program 140 including algorithm 152 and controls 160 of user interface 142, operate the LED and preferably camera 124 including microscopic zoom lens via recognition of a tag present on the reactive surface of the channel before capturing digital images of the thrombus formation under light microscopy. Alternatively, light microscope 120 is operated by computer 136 to bring the magnification and resolution of the thrombus formation into focus and coupled camera 124 captures the digital data image. The computer 136 and program 140 can additionally be configured to translate socket 38 in order to bring the thrombus formation into focus for imaging. Camera 124 can be employed with a frame rate of about 2 frames per second to capture a time-lapse image of thrombus formation. The imaging assembly 15 can take an image of thrombus formation at various points along the longitudinal axis A-A of channel 18. The time-lapse digital image data is then stored to a read/write recording medium, for example, the data storage device 137. Member 12 can then be removed from socket 38 and can be replaced by a new member 12 for running a new assay.

Once again, the user using the computer 136 having software program 140, algorithm 138 and user interface 142 can select the digital image data files for analysis. The program 140 uses the algorithm 138 to process the digital image data so as to generate the pixel data. For each digital data image, mean pixel values, mean pixel intensities are determined and the values are displayed as outputs 146, 148. A graphic of the thrombus formation is provided in display 144 of user interface 142. The pixel data is correlated to the volume of thrombus formation and reported to the user for use in adjusting the anti-thrombogenic therapy.

In one embodiment of analyzer 16, the processor 132 or computer 136 can be configured to utilize available conventional software applications capable of reading a digital data image and converting it to visual scale data. The visual scale data can be further correlated to the quantifiable aspects of thrombus formation. For example, computer 136 can be configured to run a software application 140 capable of reading static digital image data and converting it to mean grayscale data, where the mean grayscale data is a measure of intensity or darkness of the blood sample imaged in the channel 18. Any scale can by used to measure the intensity or darkness, for example, a mean grayscale can range from zero to about 255, wherein zero is black and 255 is white. Digital image data read to have a low mean grayscale score can indicate the presence of thrombus formation. Alternatively, the grayscale may be applied inversely such that a high grayscale score indicates thrombus formation. Software application 140 can be commercially available software, for example, PHOTOSHOP™, configured to run on a processor 132 or computer 136. Alternatively, grayscale level measurements may be performed manually. Shown in FIGS. 4A-4B are sample graphical displays correlating mean gray level to Integrilin concentrations and mean thrombus volume respectively using static imaging. Shown in FIG. 4C are sample static grayscale images of thrombus formations.

In addition or alternatively to the camera 124, a non-imaging photodetector 127 can be provided to pick up the fluorescence intensity from aggregated platelets in the channel 18 to generate an electrical signal. The signal from the photodetector 127 can be read by the computer 136 having software 140 with imaging algorithm 180 for correlating the fluorescence non-imaging data to the temporal evolution of the volume of thrombus formation or any other temporal and quantifiable characteristic of the thrombus formation. Moreover, the user can use interface 142 to graphically display the fluorescence data correlated to the quantifiable attributes of the thrombus formation, for example such as the graph shown in FIG. 1A.

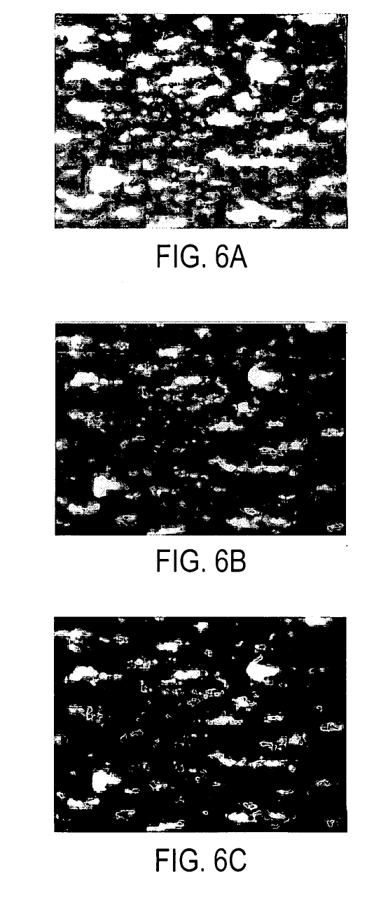

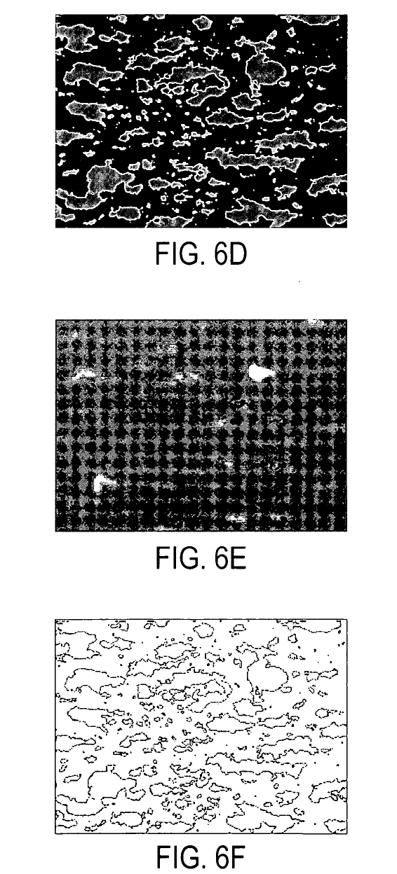

Preferably, photodetector 127 is configured with computer 136 so as to capture time-lapse or temporal evolution images of light emitted from thrombus formation, coagulation or any cellular movement in member 12 and display the image as a digital image data on a frame by frame basis, for example, as shown in FIG. 6A of a blood sample treated with a P2Y₁₂ antagonist. Algorithm 180 is preferably configured to read a single frame of displayed digital image data from photodetector 127 as an array of pixels, for example 1024×768 pixels, each pixel having a quantifiable pixel intensity. Because of the relative position of the photodetector 127 to the microscope objective of microscope 120 in imaging assembly 15, light emitted from the thrombus formation in member 12 and received by the photodetector 127 becomes diffused and appears as background. As a result, algorithm 180 includes a first aspect or background subtraction step 182 for removing the background image so as to isolate the thrombus image for quantifiable measurement. A sample resultant digital image subjected to the subtracted step 182 is shown in FIG. 6B.

In subtraction step 182, the 1024×768 array of pixels is preferably divided into a subsection array of pixels, for example, a subsection array of 32×32 pixels. For each subsection of the array, a minimum value of pixel intensity is determined. This minimum value defines the background intensity of the subsection array. In order to reduce or eliminate the noise content of the digital image, each subsection is subjected to a low-pass filtering process. The low-pass filter preferably includes a cut-off frequency of 30% the maximal spatial frequency contained in the image data. A threshold is determined for the low-pass filtered image of each subsection. More specifically, any pixels having an intensity of less than a given value corresponding to adherence of a platelet, for example 10, are preferably set to zero. A sample resultant digital image subjected to the low-pass filter process is shown in FIG. 6C.

The imaging algorithm 180 includes a second aspect or area calculation 184. Following determination of the threshold for each subsection, area calculation 184 includes taking the balance of pixels with an intensity greater than zero and resetting their intensity value preferably to one. The sum of the pixels in the subsection array define the thrombus area in units of (pixel dimension). A sample resultant digital image showing a balance of pixels set at a common pixel intensity value of, for example, one for thrombus area calculation 184 is seen in FIG. 6D.

The imaging algorithm 180 includes a third aspect or volume calculation 186. Following determination of the threshold for each subsection, volume calculation 186 includes taking the balance of pixels with an intensity greater than zero and taking the summation of those intensity values to define a thrombus volume measured in (pixel dimension)²×pixel intensity. Dividing the thrombus volume by the thrombus area can provide a mean thrombus height value. FIG. 6E is a sample resultant digital image following the threshold determination with the remaining pixels having a pixel intensity value greater than, for example, ten for thrombus volume calculation 186.

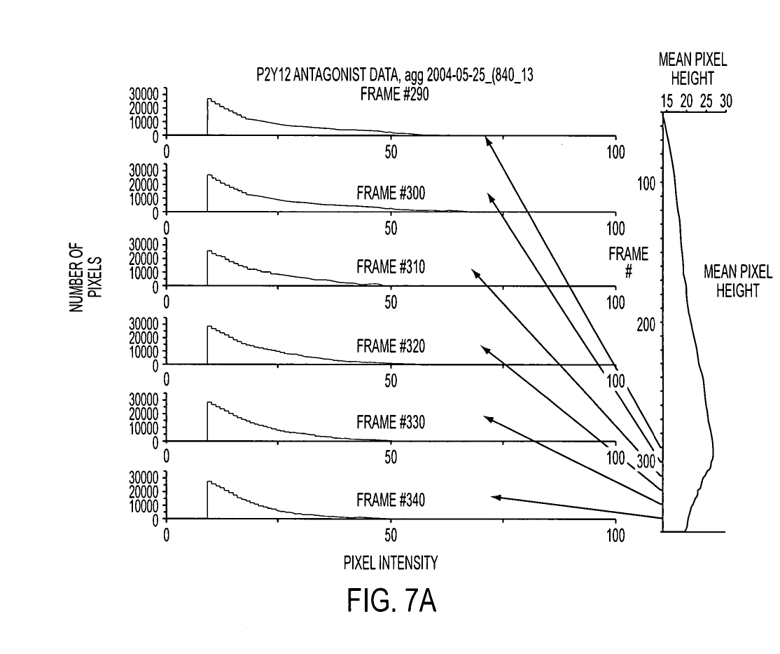

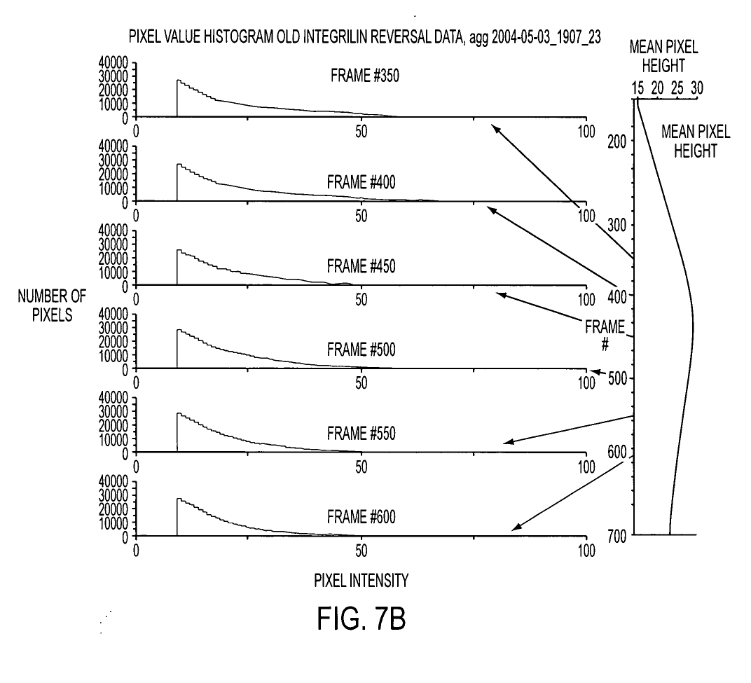

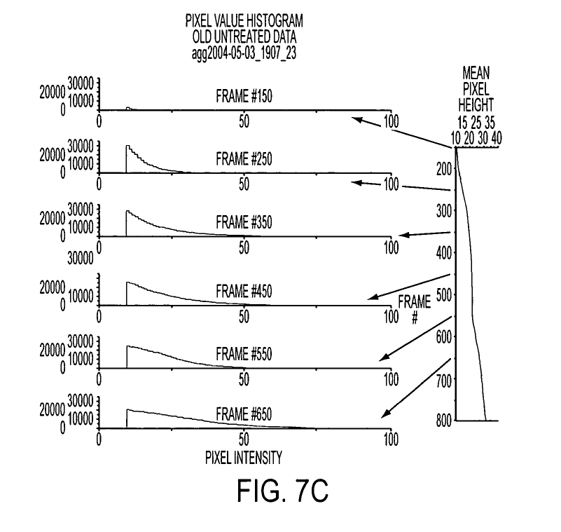

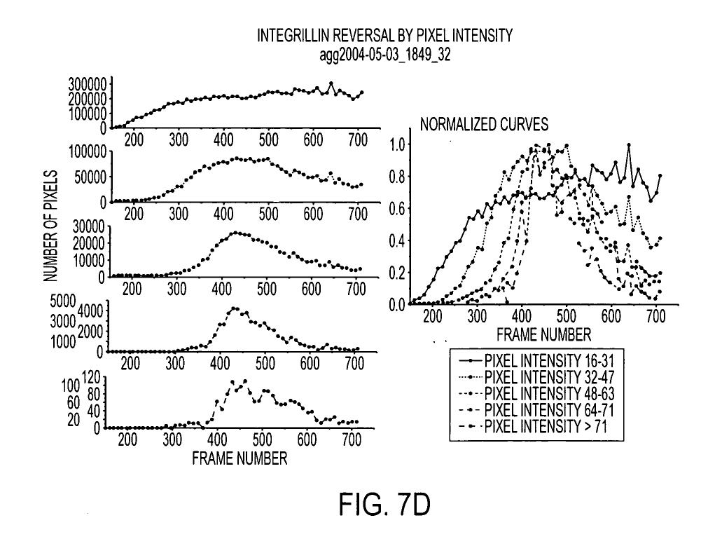

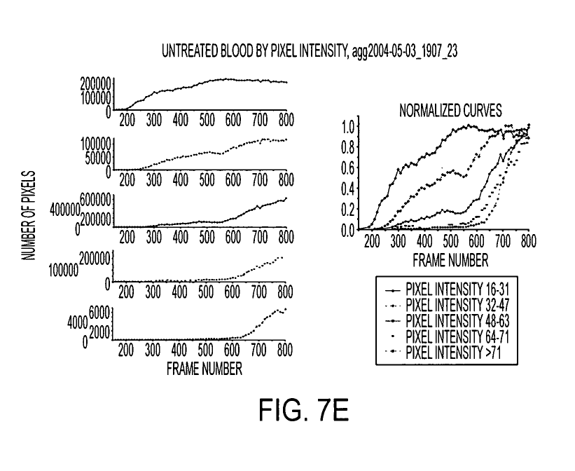

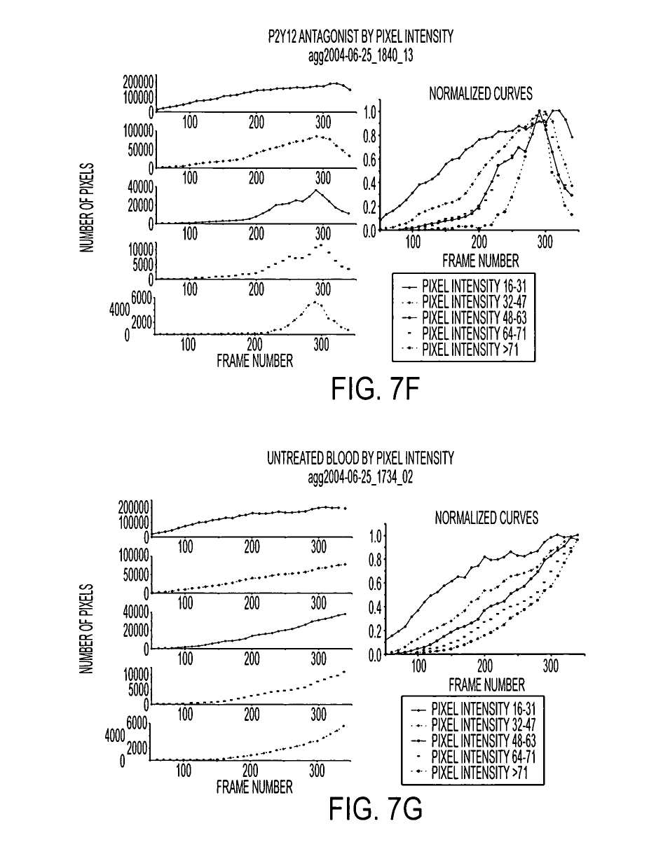

Shown in FIGS. 7A-7C are exemplary histograms of various frames of digital image data, i.e., frames 290-340, showing pixel intensity versus number of pixels. Specifically, histograms of FIGS. 7A-7C were plotted with the data derived from the volume calculation 184 for various samples of untreated and treated blood, for example, blood treated with Integrilin. Looking more specifically at the histogram of mean pixel height in FIGS. 7A-7C, pixels with higher intensity values correspond to a high thrombus formation, and increasing number of pixels at a high pixel intensity corresponds to a number of thick thrombi. The histograms and underlying digital data can be further analyzed by viewing the temporal change for a range of pixel intensity values versus the number of pixels at that intensity value from frame to frame. Sample plots of these time lapse are shown in FIGS. 7D-7G.

The imaging algorithm 180 includes a fourth aspect or perimeter calculation 188. Following determination of the area calculation 184, perimeter calculation includes taking the image of pixels, each having an intensity of one, and passing it through a high-pass filtering process. The high-pass filter includes a cut-off frequency of preferably about 50% of the maximum spatial frequency contained in the threshold image. Combining the perimeter calculation 188 with the area calculation 184 can provide information about the shape of the thrombus formation. Referring now to FIG. 6F, shown is a sample resultant digital image in which the image of FIG. 6D is subjected to the high-pass filtering process for thrombus perimeter calculation 188.

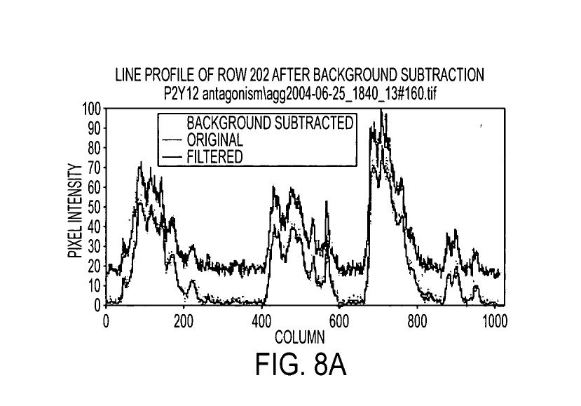

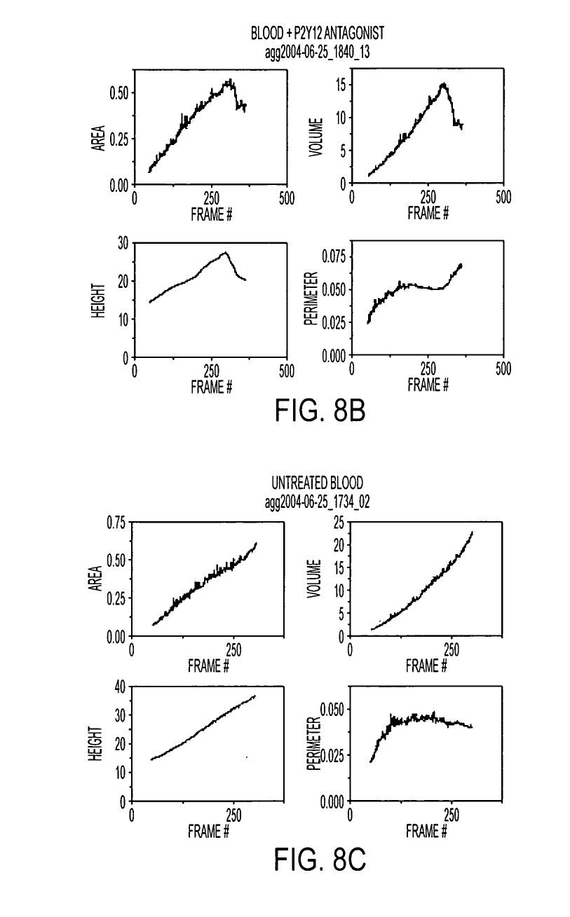

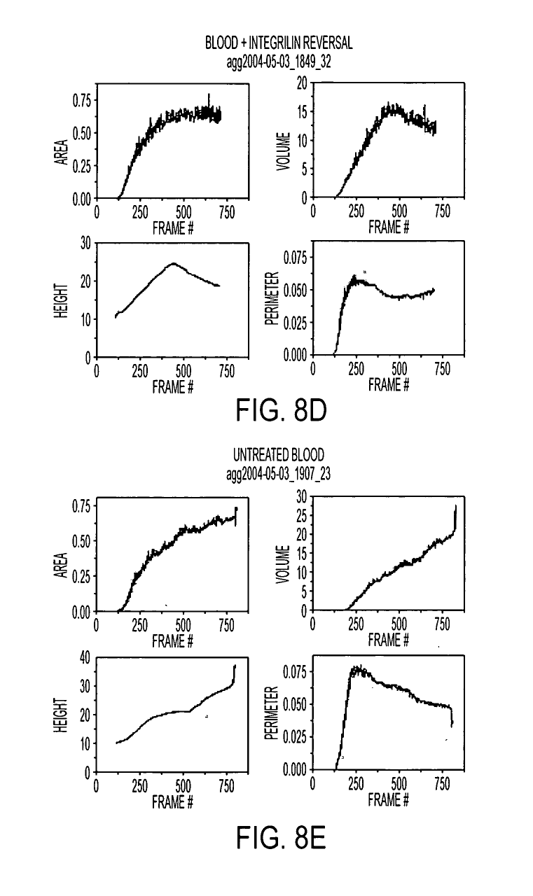

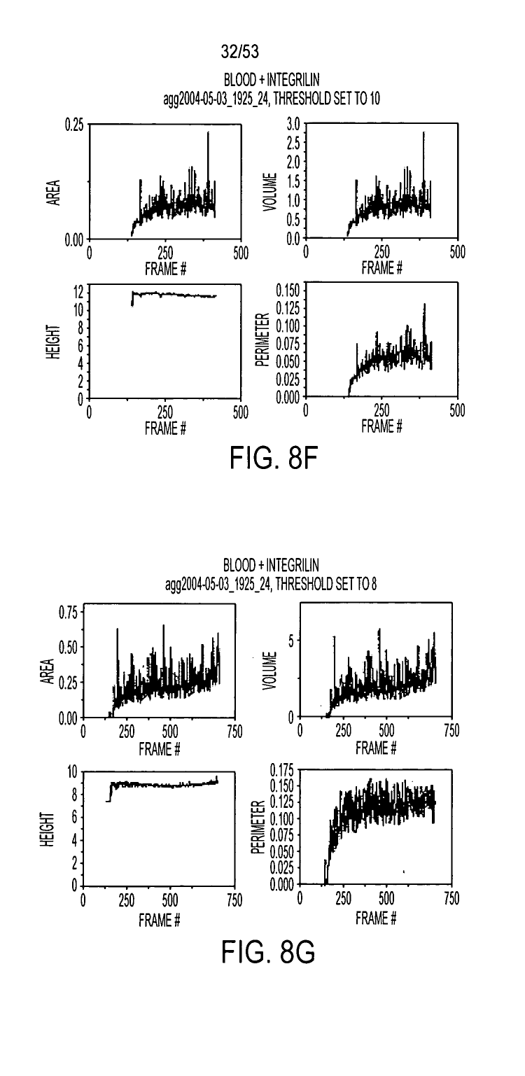

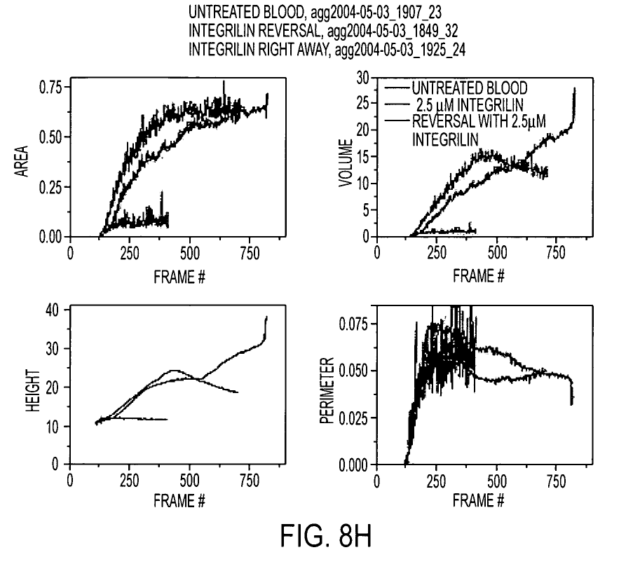

Shown are exemplary plots of pixel intensity for a single frame of digital image data in FIG. 8A and thrombus area calculation 184, thrombus volume calculation 186, thrombus height and thrombus perimeter calculation 188 for sample of treated and untreated blood in FIGS. 8B-8H each derived from the application of imaging algorithm 180. Specifically, FIG. 8B shows area, volume, height and volume plots on a time-lapse frame by frame basis for a blood sample treated with P2Y₁₂ antagonist. FIG. 8C shows area, volume, height and volume plots on a time-lapse frame by frame basis for an untreated blood sample. FIGS. 8D-8E show area, volume, height and volume plots on a time-lapse frame by frame basis for a blood sample treated with Integrilin after initial thrombus formation contrasted to a sample with no treatment. FIGS. 8F-8G show area, volume, height and volume plots on a time-lapse frame by frame basis for a blood sample pre-treated with Integrilin and a threshold pixel intensity value of ten contrasted to a sample pre-treated with Integrilin and a threshold pixel intensity value of eight. Shown in FIG. 8H are area, volume, height and volume plots overlaid upon one another on a time-lapse frame by frame basis for comparing thrombus formation in blood samples untreated, treated with Integrilin reversal and treated with Integrilin immediately after perfusion.

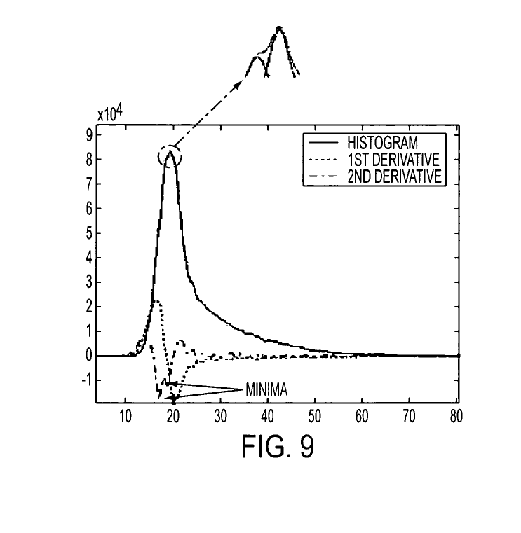

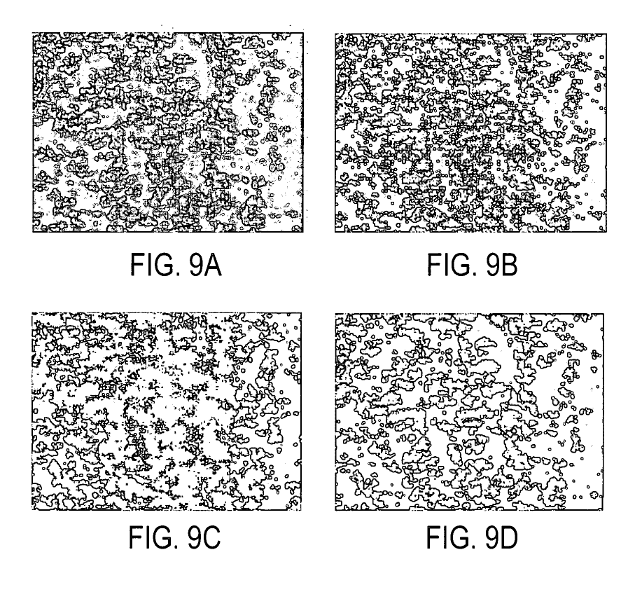

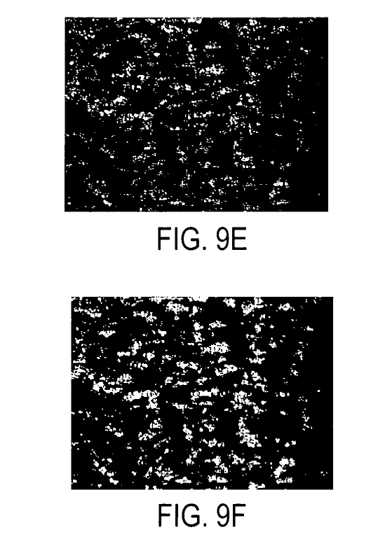

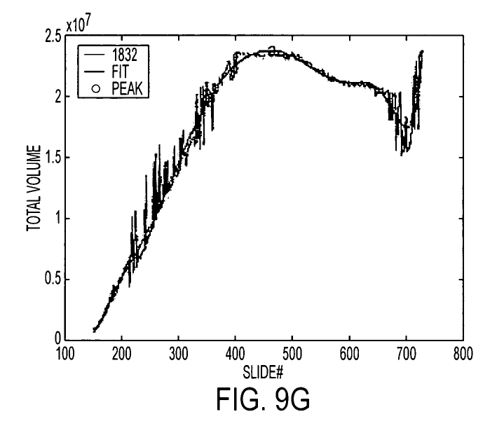

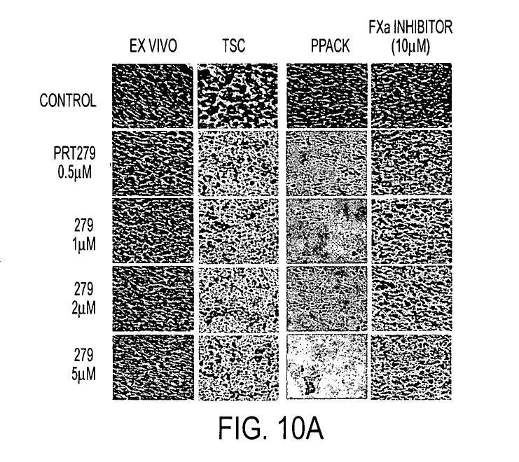

In an alternative of embodiment imaging algorithm 180, imaging algorithm 180′ can include a first aspect or segmentation process 182′, and second aspect or noise reduction process 184′, and a third aspect or watershed separation process 186′. Wherein photodetector 127 preferably produces a grayscale digital image data composed of pixels of varying pixel intensity, segmentation process 182′ which includes binarizing the grayscale digital image by producing a histogram for a single frame of data showing pixel intensity versus number of pixels. Taking the first derivative, second derivative or percentile method of the histogram of each image locates discrete peaks in the plot as shown in the plot of FIG. 9. More specifically, taking the second derivative of the initial histogram plot can reveal at least two minima points, although more are possible, wherein the first or lower minimum defining a threshold pixel intensity value. The threshold value further defines a cut-off for which pixels having an intensity less than the threshold value form the background of the digital image and the remaining foreground define the thrombus formation.

Alternative methods of computing the threshold can be utilized in which a threshold value is applied to all the images generated by the experiment. For example, the threshold value can be determined for all the images using Otsu's method (bimodal with equal variance), Kapur, Sahoo & Wong's method (1D entropy), or Abutaleb's method (2D entropy). For each of these methods, the threshold value was computed for the entire run of the experiment and then Gaussian smoothing was applied before the threshold was applied to the corresponding images.

Referring to FIG. 9, shown is the first derivative of the histogram. The zero crossing point in the first derivative is where the peak is located in the histogram. Since the histogram of the thrombus formation images produce one major peak, meaning the background and foreground peaks are overlapped, the first peak in the first derivative is selected as a threshold. This peak is located halfway between the maximum of the histogram and the lowest value of the histogram. Alternatively, using the percentile method, the threshold value can be computed by delineating, for example, 10% of the histogram as background and the upper 90% as foreground.

With the threshold determined, the noise reduction process 184′ includes a first morphological operation 190 in which small objects, for example, 5 pixels in width, that appear in the image close together, for example, within a distance of 2 pixels between each other, the objects are merged together as seen FIG. 9B. Next, the resultant image is subjected to a second morphological operator 192 in which isolated voids appearing as white pixels are removed as seen in FIG. 9C. In addition or alternatively to, small objects appearing within larger objects of the digital image data are subject to a logical operation in which pixels of the original digital image data and the digital image data produced by the first and second morphological operations 190, 192 are ANDed to produce a single image. The resultant image is smoothed by a median filter so as to define a final threshold mask shown in FIG. 9D.

The original digital image is modified by subtracting the threshold intensity value from all the pixels and applying the threshold mask to the image, thereby discarding background pixels. The resultant image is shown in FIG. 9E.

The watershed separation process 186′ is applied to the resultant image, for example the image shown in FIG. 9E, so as to identify the individual thrombi. Pixel intensity value maxima are identified and assigned a discrete color. Where discrete colors are substantially close so as to appear to merge a digital divider is located therebetween to partition the digital images of individual thrombi. The watershed is analogized to a flooding simulation. The digital image is turned upside-down, so that intensity maxima correspond to watershed minima. Modeling the image as a plastic surface, the watershed minima are imagined to define small pools in the surface with small holes in them. Imagining that the surface is submerged in water with water entering the holes such that the water level rises in the pool. Each individual pool is isolated by a dam, and anytime the pool threatens to overflow and merge with another, a dam is built to contain the overflow. As seen in the image of FIG. 9F digital “dams” or dividers are built up to partition the individual thrombus formations.

Having identified the individual thrombi, thrombus area, volume, and perimeter can be determined. For a given image, the thrombus area is obtained by counting the number of pixels forming the individual thrombi, the volume is obtained by summing the pixel intensity values for an individual thrombi, the perimeter can be obtained by counting the number of pixels that are on the edges of the thrombi. A time-lapse frame by frame plot of thrombi growth/decay can be provided by fitting the volume data to a 10th degree polynomial to display the thrombi quantities as shown in FIG. 9G.

In an alternate method in which the thrombus formation is to be imaged using fixed end point measurement imaging, a sample of blood, preferably non-anticoagulated blood, is provided for moving through member 12. The blood sample can be drawn from a reservoir and perfused through member 12 in a manner as previously described. Alternatively, the sample of blood can be drawn directly from a person. For example, where the blood is to be drawn directly from a person, shown in FIG. 31 is fluid handling portion 14a which can include a butterfly fitting 170 with a needle 172 for attachment to a vessel of a patient's arm. A patient can be undergoing anti-thrombotic drug treatment and can be hooked up to the instrument 10 to monitor thrombosis in the patient's blood. For example, the patient can be given a dose of medication and then immediately following the dosage, blood can be perfused through system 10 to determine whether the amount of medication is appropriate. Preferably and schematically shown in FIG. 1D, fluid handling portion 14a can include the requisite tubing and fittings to draw blood from a reservoir collection vessel (not shown) in a manner well known in the art.

Once the perfusion of the blood sample through the channel 18 is complete, the thrombus formation can be fixed and stained for microscopy imaging. Preferably, fluid handling portion 14b in FIG. 1D draws imaging enhancing agents from a source (not shown). For example, the thrombus formation may be rinsed and then fixed using a solution of either PBS, glutaraldehyde 2.5% or PBS, PFA 4%. The fluid handling portion 14b can apply a toluidin blue solution to stain the thrombus formation and repeatedly rinse the channel 18 with the a rinsing buffer. The member 12 is then prepared for imaging of the thrombus formation.

Member 12 is preferably maintained in socket 38 of instrument 10 for microscopy imaging by the imaging assembly 15 in accordance with the light microscopy techniques using K \overline{h}ler Illumination. As previously described, computer 136 having software program 140 including algorithm 152 and controls 160 of user interface 142 can translate the socket 38 and operate the LED 122 and camera 124 including microscopic zoom lens or alternatively interfaced microscope 120 to focus and capture fixed end point digital images of the thrombus formation. The user using the computer 136 having software program 140, algorithm 138 and user interface 142 can select the digital image data files for analysis. The program 140 uses the digital image data in the algorithm 138 to generate the pixel data. For each digital data image, mean pixel values, mean pixel intensities are determined and the values are displayed as outputs 146, 148. A graphic of the thrombus formation is provided in display 144 of user interface 142. The pixel data is correlated to the volume of thrombus formation and reported to the user for use in adjusting the anti-thrombogenic therapy.

### IV. EXAMPLES

### Example 1

### A Method to Detect the Kinetics of Thrombosis; Choice of the Anticoagulant

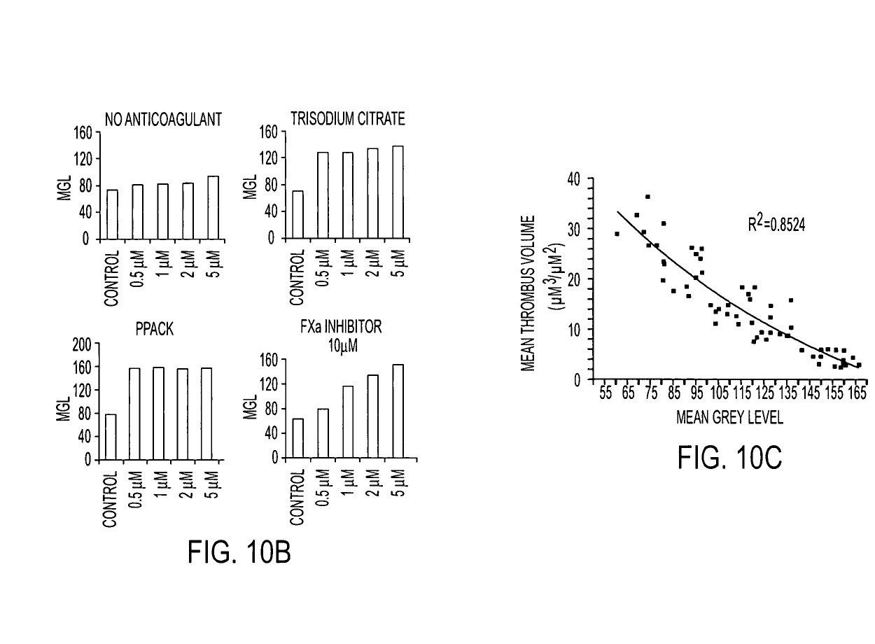

Antithrombotic activity of antiplatelet agents is artificially improved by the use of anticoagulants (see Andre et al. (2003) Circulation 108, 2697-2703). Several anticoagulants have been studied for their effects on the antithrombotic activity of a proprietary direct P2Y₁₂ antagonist in the perfusion chamber assay. Whole blood was perfused over type III collagen-coated capillaries for 4 minutes at 1000/sec. At the end of the experiment, thrombotic deposits were rinsed, fixed and stained with toluidine blue for measurement of thrombus size. Factor Xa inhibitors (and direct thrombin inhibitors like hirudin) have the least impact on the antithrombotic activity of P2Y₁₂ antagonist. It is expected that Corn Trypsin Inhibitor (which shut down contact activation pathway of coagulation) will provide similar profile. Citrate and PPACK artificially increased the antithrombotic effects of P2Y₁₂ antagonist (FIG. 10A,B). FIG. 10B represents the mean grey level (MGL) of the thrombi present in the area of observation located at 8 mm from the proximal part of the capillary. Corresponding thrombus volume was determined using the graph presented on FIG. 10C. Nonanticoagulated or Factor Xa anticoagulated human blood was perfused through type III collagen-coated capillary chambers (Vitrocom, glass rectangular capillaries 0.2 by 2 mm section) at 1500/sec for 4 minutes. After staining of the thrombotic deposits with toluidine blue for 45 seconds, an En Face picture located 8 mm downstream of the proximal part of the capillary was taken. Measurement of the gray level of each thrombus or platelets located in a window 400 μm long×250 μm wide was performed, and results are expressed as mean ±SEM using the Simple PCI software (Compix Inc Imaging System). Measurement of the mean thrombus volume (μm³/μm²) was performed at the same location on cross sections of the thrombotic deposits after epon embedding as described by Andre et al. (2003) Circulation 108, 2697-2703. Mean thrombus volume was expressed by use of Simple PCI software and plotted against the corresponding mean grey level. By using data from human in vitro (by titrating with the GPIIb/IIIa antagonist eptifibatide) experiments, a thrombotic profile (FIG. 10C) that was then used for a rapid measurement of the thrombus volume in subsequent experiments Was established

### Example 2

### A Method to Monitor in Real Time the Kinetics of Thrombosis

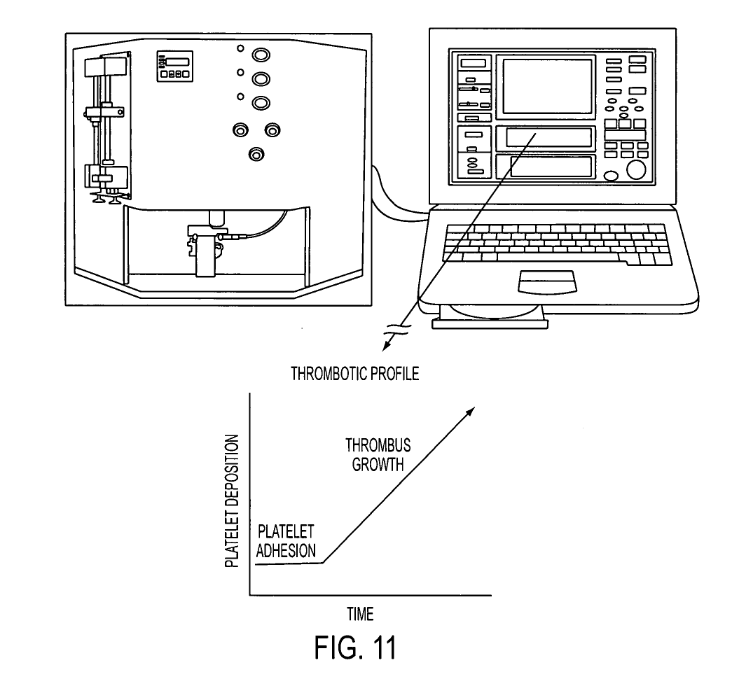

The methodology and device described herein allows the monitoring in real time of the deposition of fluorescently labeled platelets into a transparent perfusion chamber (FIG. 11). The thrombosis profiler consists of a custom built epifluorescence microscope to monitor thrombus formation and a syringe pump to establish the desired flow and wall shear rate in the capillary perfusion chamber. A thermostatic sample compartment maintains the blood sample at a temperature of 37° C. Platelets are labeled by adding an aliquot of Rhodamine 6G (final concentration 1.25 μg/ml) to whole blood. The dye is excited with light from a high-power light emitting diode with a spectral maximum at 530 nm and a spectral half width of 35 nm (Luxeon V, Lumileds Lighting, San Jose, Calif.). Excitation and emission light are filtered with a set of fluorescence filters (31002, Chroma Technologies, Rockingham, Vt.). A microscope objective images an area of 360×270 μm² on the internal wall of the capillary onto a Sony XCD X-710 digital camera (resulting magnification ca. 13×). Images are recorded at a frequency of 1 Hz. Blood flow is established by a syringe pump withdrawing blood through the capillary (Harvard Apparatus, Holliston, Mass.). A personal computer with custom software is used to control the camera and the syringe pump, and to display and record images and experimental conditions.

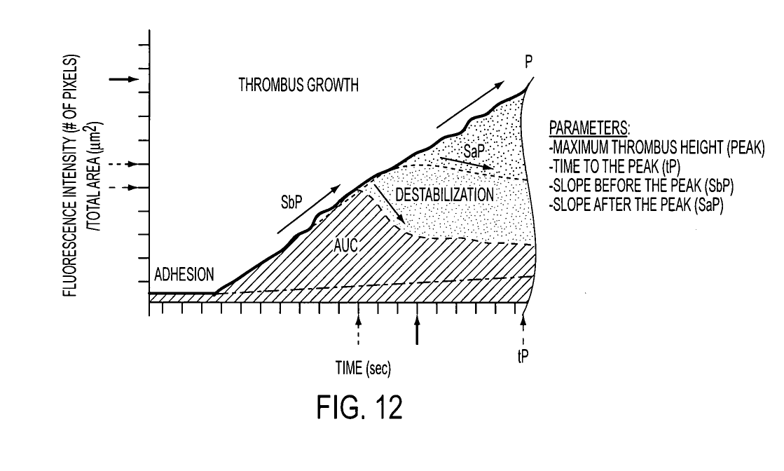

A software/algorithm has been developed in order to obtain a more representative read out of the thrombus formation over time. Although the fluorescence intensity parallels the amount of platelets deposited into the perfusion chamber, it does not distinguish platelet adhesion from thrombus volume. Since the use of antithrombotic drugs can increase platelet adhesion, thrombus size was represented as the measurement of the fluorescence intensity divided by total area (FIG. 12). Segmentation, partitioning of an image into non-overlapping regions, was accomplished based on a method proposed by Otsu (Otsu (1979) IEEE Trans. Syst. Man Cybem. 9, 62-66). This algorithm locates a point in the histogram to minimize the intra-class variance of the foreground and the background. Once the threshold is determined, pixels with values lower than the threshold are classified as background and pixels with values greater than the threshold are marked as foreground. The success of this thresholding method centers upon whether the proper threshold exists and whether it can be inferred from the image histogram. If, for example, the surface reflectance of the objects to be segmented is not distinct from the background or if the scene is not evenly illuminated then the resulting image histogram would not produce a bimodal or multi-modal graph to allow the computation of best possible threshold. For this reason we adopted a multi-stage segmentation process. Thus, after applying the threshold to generate a binary image, morphological operation “closing” (dilation followed by erosion—used to fill in holes and small gaps) followed by morphological operation “opening” (erosion followed by a dilation-used to eliminate all pixels in regions that are too small to contain the structuring element) is applied to join together the thrombi objects and clear the image of small artifacts. Next, a median filter is applied to further reduce the salt-and-pepper noise while simultaneously preserving the edges. Lastly, watershed algorithm (Gonzalez et al. (2003) Digital Image Processing, Prentice Hall) is applied to identify individual thrombi in the image. Once the image is segmented, total object volume, area and perimeter are computed. Total volume is computed as sum of intensity values of pixels inside the foreground objects. Total area is computed as number of pixels inside the foreground objects.

### Example 3

### A Method to Detect the Effect of Shear Rates on the Kinetics of Thrombosis

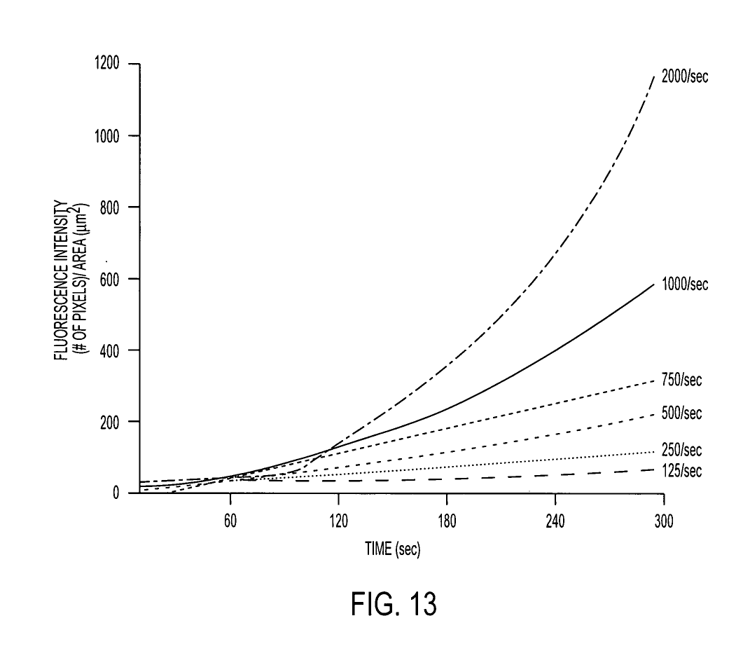

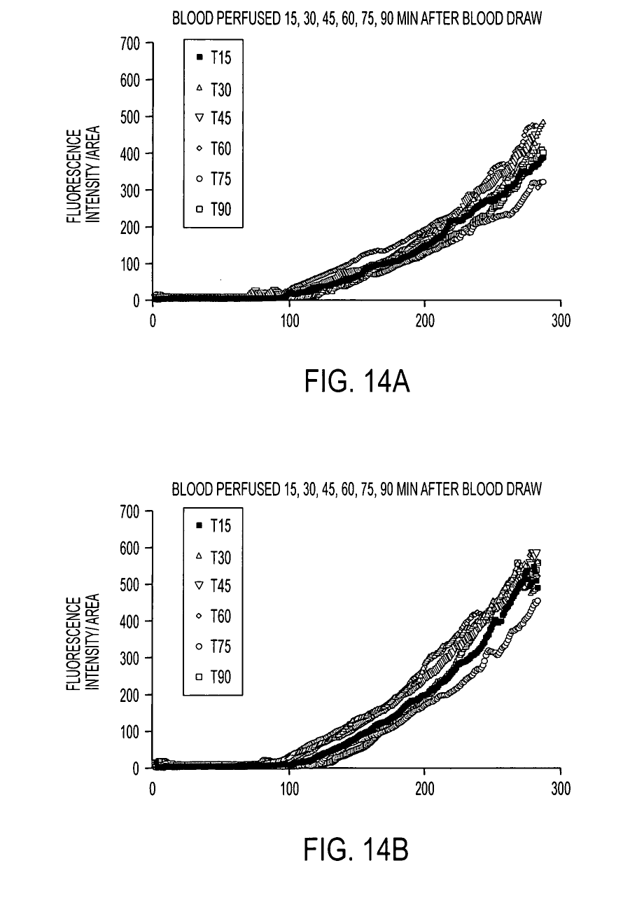

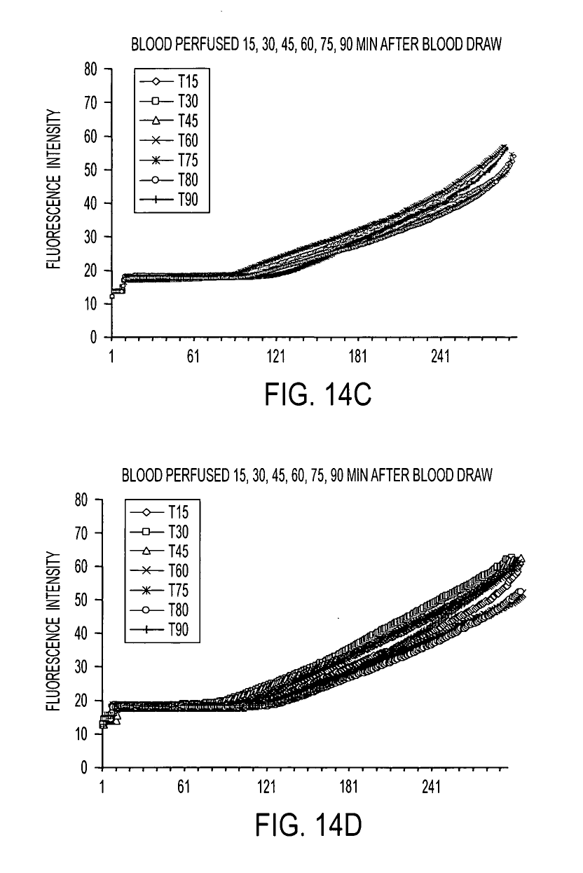

Whole blood is collected using a butterfly needle (avoid the use of vacutainer which activates platelets via high shear). Factor Xa inhibitor anticoagulated whole blood was collected from one donor. Six experiments were successively performed at increasing shear rates (from 125/sec to 2000/sec). The increase in shear rates leads to an exponential increase in platelet deposition when whole blood is perfused through a human type III collagen coated perfusion chamber (FIG. 13). FIG. 14 indicates the variability in thrombotic profiles between perfusion chambers for the same blood donor. Whole blood (anticoagulated with a factor Xa inhibitor) from one blood donor is perfused for 5 min through a collagen-coated capillary perfusion chamber at 1000/sec 15, 30, 45, 60, 75 and 90 minutes after blood has been collected. Four individual donors were studied. Experiments demonstrated reproducibility in the kinetics of the thrombotic process between different capillaries and time after blood collection. A reproducible thrombotic profile is achievable 20 minutes after blood draw and up to 70 minutes post blood draw.

### Example 4

### A Method to Characterize the Antithrombotic Activity of Antiplatelet Drugs; Inhibitors of Platelet Adhesion

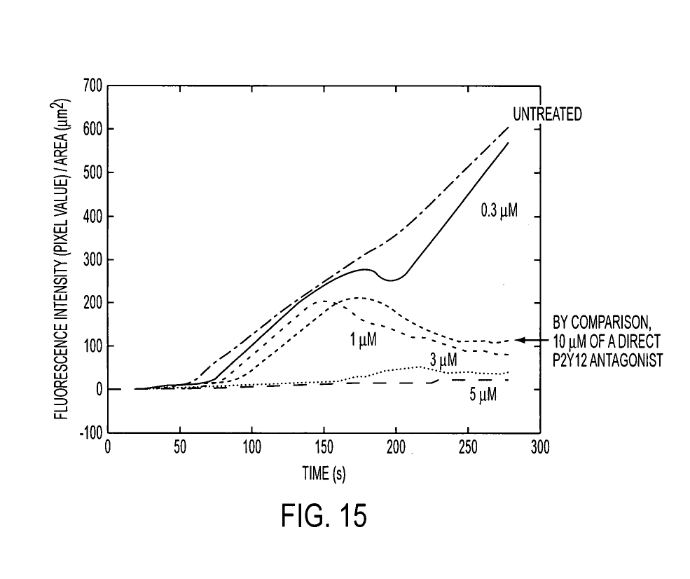

GPVI is considered to be the collagen receptor mediating platelet activation upon binding of the platelet to collagen under arterial shear rates. Signal originating from engagement of GPVI by collagen is known to be dependent upon the phosphorylation of the syk tyrosine kinase. Inhibition of Syk tyrosine kinase inhibits the platelet deposition (both thrombus formation and platelet adhesion) on fibrillar collagen in a dose dependent manner (FIG. 15). Since animals deficient in syk kinase do not exhibit a profound diathesis it is expected that a modulation of syk will be a potent and safe antithrombotic strategy.

### Example 5

### A Method to Characterize the Antithrombotic Activity of Antiplatelet Drugs; Inhibitors of Thrombus Growth

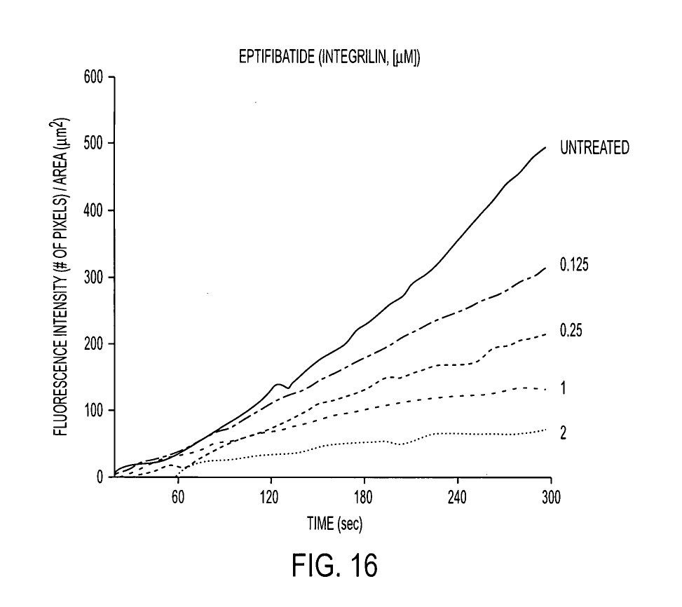

Increasing concentrations of a GP IIb/IIIa antagonist (Integrilin) were evaluated for their ability to interfere with the thrombotic process. Integrilin (spiked into Factor Xa-anticoagulated blood) dose-dependently inhibited the thrombotic process triggered by type III collagen at 1000/sec, and reached a maximum level of inhibition at the therapeutic dose (2 μM) (FIG. 16).

### Example 6

### A Method to Characterize the Antithrombotic Activity of Antiplatelet Drugs; Inhibitors of Thrombus Stability

We describe herein that inhibitors of thromboxane production (aspirin, via irreversible acetylation of Cox-1), thromboxane receptor antagonist (e.g. Ifetroban), and direct P2Y₁₂ antagonist (e.g. 2MesAMP) or prodrug that irreversibly block the P2Y₁₂ receptor (Plavix, clopidogrel) affect thrombosis via a mechanism targeting the thrombus stability. In addition, upon combination therapy, destabilization activities synergize to dramatically affect thrombus stability.

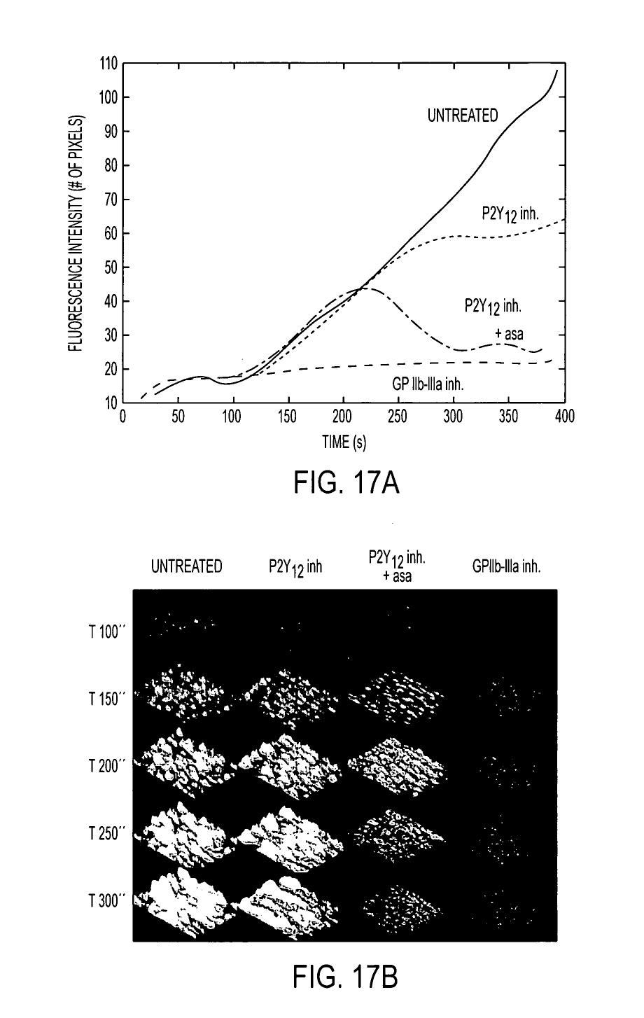

FIG. 17 shows examples of thrombotic profiles of an individual investigated before and after Plavix therapy (2 weeks at 75 mg/d), Plavix (75 mg/d for 1 week)+aspirin (325 mg/d for 1 week) and in presence of a GPIIb/IIIa inhibitor (spiked in vitro into the whole blood).

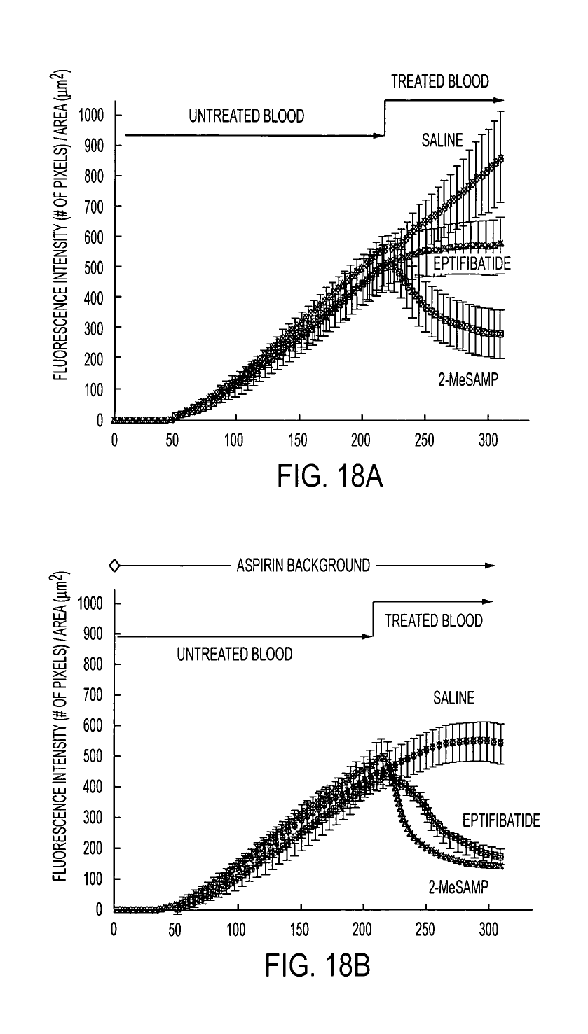

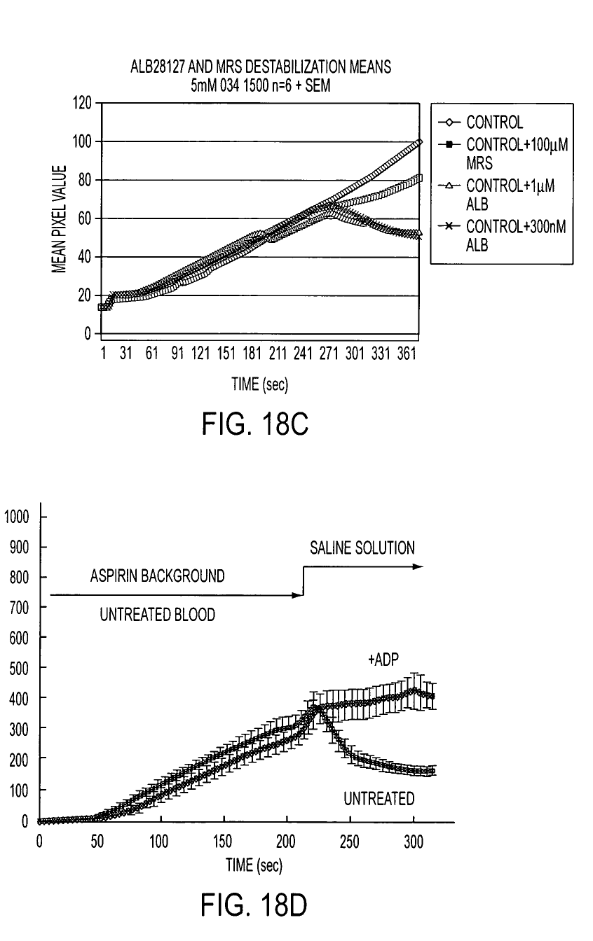

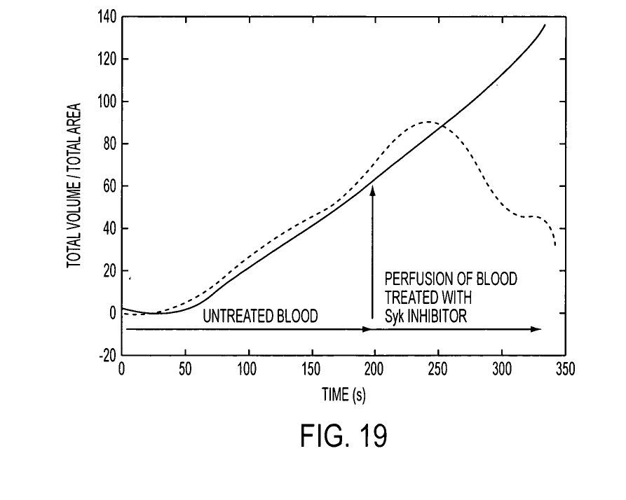

FIG. 18 shows that P2Y₁₂ inhibition (with the use of a direct acting P2Y₁₂ antagonist 2MeSAMP at 100 uM) induces the destabilization of preformed thrombi. The extent of the reversal phenomenon was increased in presence of aspirin and could not be reproduced with a GP IIb/IIIa inhibitor unless the blood donors were pretreated with aspirin (FIG. 18B). FIG. 18C shows curves of mean pixels intensity plotted over time of thrombotic profiles generated upon perfusion of blood over collagen surface under arterial shear rates. Addition of blood treated with a P2Y1 antagonist (MRS2179 at 100 uM) reduced the slope of thrombus growth but did not induced thrombus reversal, whereas the addition of a thromboxane receptor antagonist (Albany/Ifetroban at 300 nM and 1 uM) to preformed thrombi significantly altered their stability. FIG. 18D shows that a constant interaction between ADP and its receptor (P2Y₁₂) is necessary to maintain thrombus stability. Such assays can be used to detect antithrombotic activity of drugs that will target known effectors of thrombus stability reported in animal models of thrombosis (CD40L, Gas6, SLAM, SAP, Ephrin). In addition, we have found that inhibition of syk tyrosine kinase (which blocks platelet adhesion on collagen) also contributes to thrombosis reversal for lower concentration range (FIG. 19), a phenomenon that may originate from the involvement of syk downstream engagement of other glycoprotein receptors on the surface of platelets (GPIb alpha and GPIIb/IIIa).

### Example 7

### A Method that Allows for Detection of Plavix Resistant Individuals on an Aspirin Background and for a Personalization of the Antithrombotic Therapy

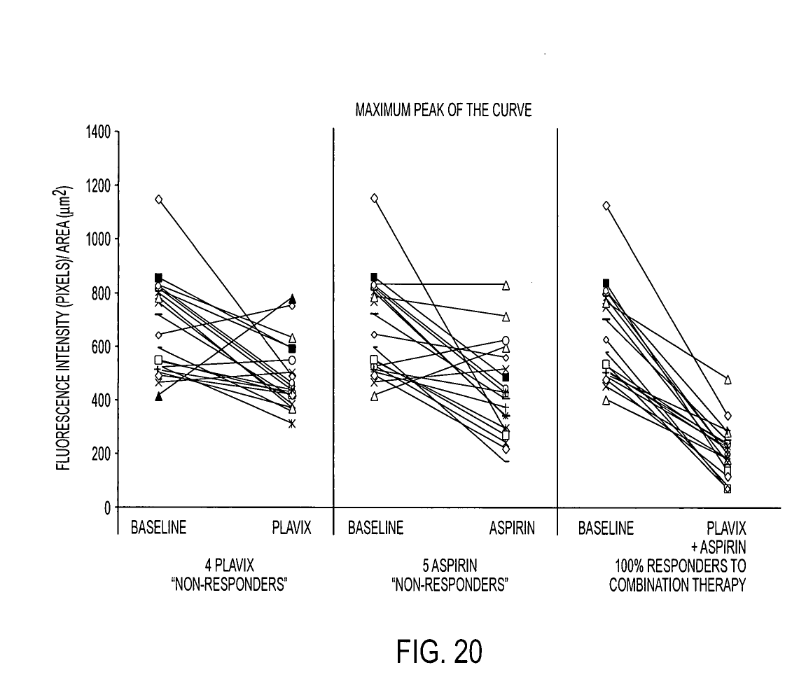

In a sequential study evaluating the thrombotic profile of 20 healthy volunteers taking successively clopidogrel (75 mg/day for 2 weeks), clopidogrel (75 mg/day)+aspirin (325 mg/day) followed by aspirin (325 mg/day), some healthy individuals did not respond to aspirin (5 out of 20) or clopidogrel monotherapy (4 out of 20 individuals) (FIG. 20) which suggested non-responsiveness. Three individuals were not benefiting from either aspirin or Plavix therapy. However, the combination of aspirin+Plavix contributed to a significant reduction in thrombus size in all patients indicating that all patients responded to both Plavix and aspirin. Thus, some patients possessed a thrombotic profile that requires a double therapy to be significantly inhibited. Therefore this method allows a personalized characterization of the thrombotic profile and the establishment of a personalized antithrombotic strategy.

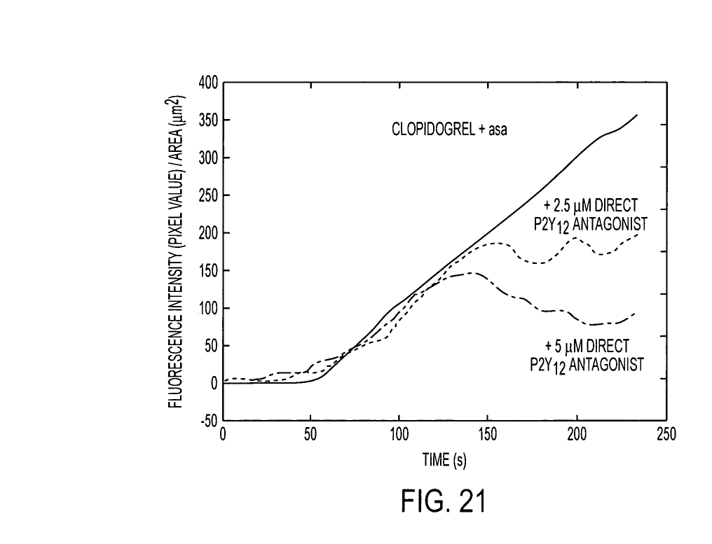

Detection of true Plavix resistance, as represented on FIG. 21, is the case of a type II Diabetic patient who was first loaded with 300 mg of clopidogrel and 325 mg aspirin. The next day, the patient received 75 mg Plavix and 325 mg aspirin. On day 2, the patient underwent PCI, was placed on Integrilin for 12 hours (infusion stopped at midnight) and receive another 300 mg dose of Clopidogrel. The thrombotic profile of the patient obtained on the morning of day 3 indicated a lack of thrombus destabilization associated with the combination therapy. The patient's stent was found occluded at noon on the same day. Thus, this method allows for determination of Plavix resistant patient and can establish the cause of the resistance (in the present case, defect in drug metabolism as a direct acting P2Y₁₂ antagonist added to the patient blood in vitro inhibited thrombosis on the Plavix background).

### Example 8

### A Method to Detect Antithrombotic Activity of Anticoagulants

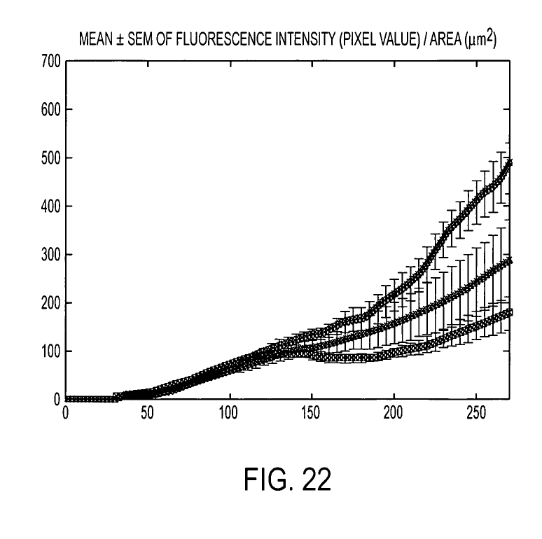

In this method, the thrombotic process can be evaluated with non-anticoagulated samples of blood. Non-anticoagulated samples of blood perfused over a thrombogenic matrix made of fibrillar collagen plus tissue factor generate thrombotic process under both venous and arterial shear rates that is sensitive to the action of different anticoagulants. In FIG. 22, the thrombotic profile, is inhibited by the therapeutic dose of enoxaparin and a factor Xa inhibitor indicating this system can be used to detect the activity of anticoagulants under arterial shear rates. Similarly, this method can be used to detect both platelet and fibrin deposition under venous shear rate conditions using for example fluorescently labeled antibodies directed against fibrin.

### Example 9

### A Method to Monitor the Pro-Inflammatory and Procoagulant Property of Adhering/Activated Platelets

Platelets adhering onto a thrombogenic surface leading to their activation lead notably to P-selectin and Phosphatidyl serine expression. P-selectin is responsible for the recruitment of leukocytes on activated/inflamed vessel wall and at sites of platelet deposition. It is known that leukocyte recruitment under these conditions will contribute to atherosclerotic plaque progression. Therefore monitoring the number of leukocyte rolling on adhering platelets could help define people at risk to develop future atherothrombotic events (number of leukocyte recruited as a predictor of future clinical events). Whole blood treated with a GPIIb/IIIa antagonist (e.g. Integrilin at the therapeutic dose 2-3 uM) and perfused over a collagen surface generate a monolayer of adhering platelets. Although thrombus formation is abrogated under these conditions, platelet activation is not affected. Two to three minutes after the start of the perfusion at arterial shear rates of about ˜600/sec, leukocytes stained with rhodamine 6G are being recruited and roll over the adhering platelets. Antithrombotic agents (or agents targeting the P-selectin/PSGL-1 pathway) that will reduce the amount of leukocyte rolling on adhering platelets will therefore potentially reduce the risks of atherothrombotic events.

### Example 10

### A Method to Detect the Hemostatic and Prothrombotic Activity of Liposomes, Blood Platelet Substitutes (Synthetic Platelets)

The methodology described herein, allows for the identification and observation of synthetic platelets or liposomes interacting with thrombogenic surfaces or surfaces presenting antibodies. Therefore, the contributions to the thrombotic or hemostatic processes of synthetic platelets or liposomes can be monitored in this assay.

### Example 11

### A Method to Detect Circulating Tumor Cells

Some circulating tumour cells are recruited on surfaces expressing P-selectin. Therefore, whole blood treated with a GPIIb/IIIa inhibitor or any other antagonist that will not affect platelet activation will provide a P-selectin enriched surface that can be utilized to observe circulating tumour cells (via staining with a specific marker of the tumour cell coupled to FITC for example) or recruit tumour cells via co-expression of P-selectin, fibronectin and presence of chemokines implicated in immigration of tumor cells. An implantable microchamber maybe utilized in order to reduce the amount of circulating tumour cells in cancer patients developing metastasis.

While the present invention has been disclosed with reference to certain embodiments, numerous modifications, alterations and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

All patents, patent applications and references referred to in this application are herein incorporated by reference in their entirety for all purposes.

What I claim is:

1. A member for capturing a component of a blood sample comprising:
a body having a channel defining a holding volume of less than about 20 μl to hold the sample, the channel having a height of less than about 1 mm.

2. A member for capturing a component of a blood sample comprising:
a body having a plurality of channels defining a total holding volume of less than about 20 μl, each channel having a height of less than about 1 mm to hold at least a portion of the sample.

3. A method of quantifying a thrombus formation, blood coagulation, inflammatory or circulating tumour cells recruitment comprising:
digitally capturing real-time thrombi formation (any blood cell deposition) in a sample of blood using a photodetector so as to generate an electrical signal;
correlating the electrical signal to a first grayscale digital image data array having pixel intensity values, the digital image data including thrombi digital data and background noise digital data;
reducing the background noise digital data to isolate the thrombi digital data including,
defining a first pixel intensity function, the first pixel intensity function being defined by the number of pixels in the array and the pixel intensity value of each pixel;
determining a threshold of the pixel intensity value by taking a second derivative of the pixel intensity function, the threshold of the pixel intensity value defining the background noise digital data and the thrombi digital data;
deleting the background noise digital data to isolate the thrombi digital data and define a second digital image data;
determining at least one discrete thrombus formation in the thrombi digital data including,
determining a second pixel intensity function from the second digital image data, the second pixel intensity function being defined by the number of pixels in the array and the pixel intensity value of each pixel;
determining maxima of the second pixel intensity function so as to determine the discrete thrombus formation; and
quantifying the discrete thrombus formation including,
counting the number of pixels defining the discrete thrombus formation so as to define an area of the thrombus formation; and
taking a sum total of the pixel intensity values for the discrete thrombus formation so as to define a volume of the thrombus formation.

4. An instrument for capturing an image of rolling, adhesion, aggregation, disaggregation in a blood sample, the instrument comprising:
a microchip member defining a longitudinal axis and having a first connecting portion, the member comprising:
a first inlet having a first interface to introduce the blood sample into the member;
at least a second inlet having a second interface to introduce an agent;
a plurality of channels along the longitudinal axis, the plurality of channels being at least partially coated with a material that induces blood circulating cells recruitment and aggregation of at least one blood component and in communication with the first and second inlets to receive and combine the blood sample and agent so as to initiate aggregation within the channels for imaging, each of the channels having an outlet to permit flow therethrough and defining a cross-sectional area perpendicular to and variable along the longitudinal axis so as to vary hemodynamic properties of the channel along the longitudinal axis;
a fluid handling assembly comprising:
a valve means interfaced with the second inlet interface to control introduction of the agent through the second inlet; and
a pump disposed relative to each outlet of plurality of chambers so as to draw the blood sample through the channel at a flow rate; and
an imaging assembly comprising:
a device that detects aggregation or any blood cell type deposition,
a stage having a second connecting portion associated with the first connecting portion to hold and dispose the microchip member relative to the device for detecting the aggregation in the plurality of channels; and
an analyzer having a first control means associated with the fluid assembly to control the valve means and the pump, the analyzer having a second control means associated with the imaging assembly to control imaging of the aggregation, the second control means including at least one algorithm to quantify at least one characteristic of the aggregation.

5. An instrument for capturing an image of thrombus formation in a blood sample, the instrument comprising:
a member for capturing the kinetics of thrombosis (adhesion, thrombus growth and stability),
a lower portion including a channel;
an upper portion being a tube member disposed within the channel, the tube member defining a longitudinal axis and having an inlet and an outlet through which the blood sample flows, the tube member further having an upper surface with an opening; and
a cover member dimensioned and configured to seal the opening, the cover member including a thrombogenic material for initiating thrombus formation in the tube member, the thrombogenic material being in communication with the blood sample when the blood sample flows through the tube member;
a fluid handling assembly including a pump disposed relative to the outlet of the tube member so as to draw the blood sample through the tube member; and
an imaging assembly comprising:
a device that detects thrombus formation;
a stage associated with the lower portion to hold and dispose the member relative to the light microscopy device for imaging the thrombus formation in the tube member;
a digital camera interfaced with the device to capture a digital image of the thrombus formation in real-time;
an analyzer having a first control means associated with the fluid assembly to control the pump, the analyzer having a second control means associated with the imaging assembly to control imaging of the thrombus formation, the second control means including at least one algorithm to quantify at least one characteristic of the thrombus formation.

6. An instrument for imaging and analyzing a reaction between a blood sample and an agent, the instrument comprising:
means for capturing the reaction including a microchip member defining a longitudinal axis and having a first interface to introduce the blood sample into the member and a second interface to introduce the agent, the member further comprising a plurality of channels along the longitudinal axis to receive and combine the blood sample and agent so as to initiate and capture the reaction within the channels for imaging, each of the plurality of channels having an outlet to permit flow therethrough and defining a cross-sectional area perpendicular to and variable along the longitudinal axis so as to vary hemodynamic properties of the channel along the longitudinal axis; and
means for imaging the reaction within the channels including a means to hold and dispose the microchip member relative to the imaging means, a means to capture the reaction in real-time; and an analyzer having a first control means associated with the capturing means to control the flow of the blood sample and the agent through the microchip member and at least one algorithm to quantify at least one characteristic of the reaction.

7. An instrument for capturing an image of thrombus formation in a blood sample, the instrument comprising:
a member defining a channel therethrough;
a fluid handling assembly that permits the blood sample to move through the channel at a flow rate; and
an imaging assembly including a microscopy device, the imaging assembly being disposed relative to the channel so as to capture light rays defining the image of thrombus formation in the channel.

8. The instrument of claim 7, wherein the microscopy device comprises a light microscope.

9. The instrument of claim 8, wherein the imaging assembly further comprises Köhler illumination optics.

10. The instrument of claim 7, wherein the imaging assembly comprises an LED to illuminate the blood sample.

11. The instrument of claim 7, wherein the imaging assembly comprises a digital camera to capture the image and convert the image to digital data.

12. The instrument of claim 7, further comprising an analyzer to quantify the volume of thrombus formation using the image.

13. The instrument of claim 12, wherein the analyzer comprises a computer having software including at least one algorithm to correlate the image to thrombus volume.

14. The instrument of claim 13, wherein the software has at least a second algorithm for controlling the fluid handling assembly to vary the flow rate of the blood sample through the channel.

15. The instrument of claim 7, wherein the member is a capillary tube.

16. The instrument of claim 7, wherein the channel defines a longitudinal axis along which the blood moves and a cross-sectional area perpendicular to the longitudinal axis.

17. The instrument of claim 16, wherein the cross-sectional area is substantially rectangular.

18. The instrument of claim 16, wherein the cross-sectional areas is substantially circular.

19. The instrument of claim 7, wherein the member comprises a transparent section defining at least one surface of the channel.

20. The instrument of claim 19, wherein the transparent section comprises a non-thrombogenic material.

21. The instrument of claim 19, wherein at least a portion of the transparent section comprises at least one thrombogenic coating.

22. The instrument of claim 7, wherein the fluid handling assembly comprises a first portion for moving the blood through the channel and a second portion to deliver an image enhancing agent to the blood sample.

23. The instrument of claim 22, wherein the first portion comprises a pump to move the blood sample through the channel, the pump having a flow regulating mechanism to regulate the flow rate of the blood through the channel.

24. The instrument of claim 23, wherein the pump is a syringe pump.

25. The instrument of claim 23, wherein the regulating mechanism comprises a computer interfaced with the pump and a software application having at least one algorithm to regulate the flow rate of blood through the channel.

26. The instrument of claim 22, wherein the second portion comprises a delivery device and a computer interfaced with the delivery device, the computer comprises software having at least one algorithm for regulating the delivery of the image enhancing agent.

27. The instrument of claim 22, wherein the second portion is in communication with the channel.

28. The instrument of claim 7, wherein the fluid handling assembly comprises a receiver to orient the member, the receiver having a first connector portion and a second connector portion; and
the member comprises an inlet end and an outlet end each in communication with the channel, the inlet end detachably connected to the first connector portion to permit the blood sample to move through the inlet end, the channel and the outlet end.

29. An instrument for capturing an image of thrombus formation in a blood sample, the instrument comprising:
means for capturing thrombus formation; and
microscopy means for capturing an image of the thrombus formation.

30. The instrument of claim 29 further comprising a means for quantifying the thrombus formation using the image.

31. A system for quantifying thrombus formation from a digital data image of a blood sample comprising:
a digital read/write medium to load the digital data;
a processor for converting the digital data to pixel data; and
software having at least one algorithm for quantifying the thrombus formation using the pixel data.

32. The system of claim 31 further comprising a display for displaying the digital data image of the blood sample.

33. The system of claim 31, wherein the algorithm determines a pixel intensity from the pixel data and correlates the pixel data to a volume of thrombus formation.

34. The system of claim 31, wherein the at least one algorithm correlates the pixel data over a period of time to a rate of thrombus formation.

35. A method of quantifying thrombus formation, blood coagulation, inflammatory and cancer cells recruitment from a blood sample comprising:
providing a member having at least one channel, the channel including at least one surface coated with a thrombogenic material;
moving the blood sample through the channel initiating thrombus formation upon the blood sample contacting the thrombogenic, pro-inflammatory, or chemo-attractant material; and
imaging the thrombus formation, or any recruitment, rolling, adhesion, aggregation of circulating cells.

36. The method of claim 35, wherein the imaging comprises using light microscopy.

37. The method of claim 35, wherein imaging the thrombus formation comprises generating a digital data image of the thrombus formation.

38. The method of claim 35, wherein the moving the blood and the imaging are performed simultaneously.

39. The method of claim 35 further comprising analyzing the digital data image to quantify the thrombus formation.

40. The method of claim 39, wherein analyzing the digital data image comprises converting the digital data to pixel data and correlating the pixel data to thrombus volume.

41. The method of claim 39, wherein analyzing the digital data image comprises converting the digital data to pixel data and correlating the pixel data to a rate of thrombus formation.

42. The method of claim 35 further comprising providing the blood sample from a patient administered with an anti-thrombotic agent.

43. A member for capturing thrombus formation comprising:
a body defining at least one channel therethrough, the channel having an inlet end and an outlet end;
a transparent section of the body defining at least a portion of the channel, the transparent portion comprising substantially a non-thrombogenic material; and
at least a portion of the transparent portion being coated with a thrombogenic material.

44. The member of claim 43, wherein the body is a microchip and the at least one channel defines a width of about 500 μm.

45. The member of claim 43, wherein the body comprises an upper body portion, a lower body portion and a tube member inserted between the upper and lower body portion.

46. An instrument for capturing an image of thrombus formation in a member having a channel for moving a blood sample therethrough, the instrument comprising:
a socket member configured to receive the member;
a fluid handling assembly that permits the blood sample to move through the channel at a flow rate; and
an imaging assembly including a microscopy device, the imaging assembling being disposed relative to the socket to permit the imaging assembly to capture an image of thrombus formation in the channel.

47. The instrument of claim 40 wherein the socket has a first portion for delivering the blood sample to the member and a second portion for delivering at least one imaging enhancing agent to the member.
